(19) United States
(12) Patent Application Publication
(10) Pub. No.: US 2022/0154273 A1
(43) Pub. Date: May 19, 2022

(54) INCORPORATION AND IMAGING MIXES

(71) Applicants: ILLUMINA, INC., San Diego, CA (US); ILLUMINA CAMBRIDGE LIMITED, Cambridge (GB)

(72) Inventors: Gianluca Andrea Artioli, Cambridge (GB); Mathieu Lessard-Viger, San Diego, CA (US); Brian D. Mather, San Diego, CA (US); Seth M. McDonald, San Diego, CA (US); Kaitlin M. Pugliese, San Diego, CA (US); Xavier von Hatten, Cambridge (GB)

(21) Appl. No.: 17/525,530

(22) Filed: Nov. 12, 2021

Related U.S. Application Data
(60) Provisional application No. 63/114,302, filed on Nov. 16, 2020.

(51) Int. Cl. C12Q 1/6874; B01L 3/00

(52) U.S. Cl. CPC C12Q 1/6874; B01L 2200/0621; B01L 2200/026; B01L 3/502738

(57) ABSTRACT

An example of an incorporation mix includes a liquid carrier, a complex, and a labeled nucleotide. The complex includes a polymerase and a plasmonic nanostructure linked to the polymerase. The labeled nucleotide includes a nucleotide, a 3′ OH blocking group attached to a sugar of the nucleotide, and a dye label attached to a base of the nucleotide.

### CROSS-REFERENCE TO RELATED APPLICATION

This application claims the benefit of U.S. Provisional Application Ser. No. 63/114,302, filed Nov. 16, 2020, the contents of which is incorporated by reference herein in its entirety.

### BACKGROUND

Various protocols in biological or chemical research involve performing a large number of controlled reactions on local support surfaces or within predefined reaction chambers. The designated reactions may then be observed or detected and subsequent analysis may help identify or reveal properties of chemicals involved in the reaction. In some examples, the controlled reactions alter charge, conductivity, or some other electrical property, and thus an electronic system may be used for detection. In other examples, the controlled reactions generate fluorescence, and thus an optical system may be used for detection.

### SUMMARY

Incorporation and imaging mixes are disclosed herein. These mixes are suitable for use with sequencing methods where nucleotide base calling is performed via the detection of optical signals. The mixes disclosed herein enhance these optical signals via plasmonic resonance and the precise positioning of plasmonic nanostructures with respect to dye labels of incorporated labeled nucleotides. Several mechanisms and techniques are disclosed herein which localize the plasmonic nanostructure within signal enhancing proximity of the dye label, which is a component of the labeled nucleotide that has been incorporated into a nascent nucleic acid strand during sequencing. The localization allows the plasmonic nanostructure to be held within signal enhancing proximity of the dye label during imaging, and thus the plasmonic nanostructure is able to enhance the optical signal of the dye label.

### INTRODUCTION

A first aspect disclosed herein is an incorporation mix comprising a liquid carrier; a complex including a polymerase and a plasmonic nanostructure linked to the polymerase; and a labeled nucleotide including a nucleotide; a 3′ OH blocking group attached to a sugar of the nucleotide; and a dye label attached to a base of the nucleotide.

In an example of the first aspect, the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

In an example of the first aspect, the plasmonic nanostructure is chemically conjugated to an amine or a cysteine of the polymerase.

In an example of the first aspect an oligonucleotide is attached to the polymerase; and the oligonucleotide is hybridized to a complementary oligonucleotide tether that is attached to the plasmonic nanostructure.

In an example of the first aspect, an oligonucleotide is attached to the polymerase; and the oligonucleotide is hybridized to a complementary portion of an oligonucleotide tether that also includes an additional portion that is wrapped around the plasmonic nanostructure.

In an example of the first aspect, the plasmonic nanostructure is functionalized with a first member of a binding pair; and the polymerase includes or is functionalized with a second member of the binding pair. In one example, the first member and the second member include a NiNTA ligand and a histidine tag, or streptavidin and biotin, or a spytag and a spycatcher, or maleimide and cysteine, or azide and dibenzocyclooctyne.

It is to be understood that any features of the first aspect may be combined together in any desirable manner and/or may be combined with any of the examples disclosed herein to achieve the benefits as described in this disclosure, including, for example, enhanced fluorescence signals during sequencing imaging.

A second aspect disclosed herein is a method comprising introducing an incorporation mix to a flow cell including clusters of template strands, the incorporation mix including a liquid carrier; a plurality of complexes, each complex including a polymerase and a plasmonic nanostructure linked to the polymerase; and a plurality of labeled nucleotides, each labeled nucleotide including a nucleotide; a 3′ OH blocking group attached to a sugar of the nucleotide; and a dye label attached to a base of the nucleotide; whereby at least one the polymerases i) incorporates an individual one of the labeled nucleotides into a nascent strand along one the template strands, and ii) maintains its linked plasmonic nanostructure within proximity of the individual one of the labeled nucleotides; and optically imaging the incorporation while the plasmonic nanostructure is maintained.

It is to be understood that any features of the second aspect may be combined together in any desirable manner. Moreover, it is to be understood that any combination of features of the first aspect and/or of the second aspect may be used together, and/or may be combined with any of the examples disclosed herein to achieve the benefits as described in this disclosure, including, for example, enhanced fluorescence signals during sequencing imaging.

A third aspect disclosed herein is a kit comprising an incorporation mix including: a liquid carrier; a polymerase; and a labeled nucleotide including a nucleotide; a 3′ OH blocking group attached to a sugar of the nucleotide; and a dye label attached to a base of the nucleotide; and an imaging mix, including a second liquid carrier; and a plasmonic nanostructure functionalized to associate itself within proximity of the labeled nucleotide after an incorporation event involving the labeled nucleotide.

In an example of the third aspect, the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

In an example of the third aspect, the plasmonic nanostructure is functionalized with a second polymerase. In one example, the plasmonic nanostructure is chemically conjugated to an amine or a cysteine of the second polymerase. In another example, an oligonucleotide is attached to the second polymerase; and the oligonucleotide is hybridized to a complementary oligonucleotide tether that is attached to the plasmonic nanostructure. In still another example, an oligonucleotide is attached to the second polymerase; and the oligonucleotide is hybridized to a complementary oligonucleotide tether that includes a portion that is wrapped around the plasmonic nanostructure.

In an example of the third aspect, the plasmonic nanostructure is functionalized with a first member of a binding pair; and the polymerase includes or is functionalized with a second member of the binding pair. In one example, the first member and the second member include a NiNTA ligand and a histidine tag, or streptavidin and biotin, or a spytag and a spycatcher, or maleimide and cysteine, or azide and dibenzocyclooctyne. In some examples, the polymerase further comprises a DNA binding domain attached to a surface thereof. In other examples, the polymerase further comprises a surface tether attached to a surface thereof; and a flow cell surface binding agent attached to the surface tether.

In an example of the third aspect, the plasmonic nanostructure is functionalized with streptavidin; and the labeled nucleotide is biotinylated.

It is to be understood that any features of the third aspect may be combined together in any desirable manner. Moreover, it is to be understood that any combination of features of the first aspect and/or of the second aspect and/or of the third aspect may be used together, and/or may be combined with any of the examples disclosed herein to achieve the benefits as described in this disclosure, including, for example, enhanced fluorescence signals during sequencing imaging.

A fourth aspect disclosed herein is a method comprising introducing an incorporation mix to a flow cell including clusters of template strands, the incorporation mix including a liquid carrier; a plurality of polymerases; and a plurality of labeled nucleotides, each labeled nucleotide including a nucleotide; a 3′ OH blocking group attached to a sugar of the nucleotide; and a dye label attached to a base of the nucleotide; whereby at least one the polymerases incorporates an individual one of the labeled nucleotides into a nascent strand along one the template strands; introducing an imaging mix into the flow cell, the imaging mix including a second liquid carrier; and a plurality of functionalized plasmonic nanostructures; whereby at least one of the functionalized plasmonic nanostructures associates itself within proximity of the individual one of the labeled nucleotides; and optically imaging the incorporation while the at least one of the functionalized plasmonic nanostructures is associated with the individual one of the labeled nucleotides.

In an example of the fourth aspect, each of the functionalized plasmonic nanostructures is functionalized with a second polymerase; and the method further comprises removing the incorporation mix prior to introducing the imaging mix.

In an example of the fourth aspect, each of the functionalized plasmonic nanostructures is functionalized with a first member of a binding pair; each of the polymerases includes a second member of the binding pair; and the method further comprises removing the incorporation mix prior to introducing the imaging mix.

In an example of the fourth aspect, each of the functionalized plasmonic nanostructures is functionalized with streptavidin; each of the labeled nucleotides is biotinylated; and the method further comprises removing the incorporation mix prior to introducing the imaging mix.

It is to be understood that any features of the fourth aspect may be combined together in any desirable manner. Moreover, it is to be understood that any combination of features of the first aspect and/or of the second aspect and/or of the third aspect and/or of the fourth aspect may be used together, and/or may be combined with any of the examples disclosed herein to achieve the benefits as described in this disclosure, including, for example, enhanced fluorescence signals during sequencing imaging.

A fifth aspect disclosed herein is a labeled nucleotide comprising a nucleotide; a 3′ OH blocking group attached to a sugar of the nucleotide; a dye label attached to a base of the nucleotide; and a plasmonic nanostructure attached to the base of the nucleotide or to the dye label.

In an example of the fifth aspect, the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

In an example of the fifth aspect, the plasmonic nanostructure is attached to the base of the nucleotide through a double stranded deoxyribonucleic acid strand.

In an example of the fifth aspect, a first linking molecule attaches the dye label to the base of the nucleotide; a second linking molecule attaches the plasmonic nanostructure to the base of the nucleotide; and the first linking molecule has a first length that is within from about 3 nm to about 12 nm of a second length of the second linking molecule.

In an example of the fifth aspect, a first linking molecule attaches the dye label to the base of the nucleotide; a second linking molecule attaches the plasmonic nanostructure to the base of the nucleotide; and the first linking molecule has a first length, the second linking molecule has a second length, and together the first and second lengths range from about 3 nm to about 12 nm.

It is to be understood that any features of the fifth aspect may be combined together in any desirable manner. Moreover, it is to be understood that any combination of features of the first aspect and/or of the second aspect and/or of the third aspect and/or of the fourth aspect and/or of the fifth aspect may be used together, and/or may be combined with any of the examples disclosed herein to achieve the benefits as described in this disclosure, including, for example, enhanced fluorescence signals during sequencing imaging.

A sixth aspect disclosed herein is a method comprising introducing an incorporation mix to a flow cell including clusters of template strands, the incorporation mix including a liquid carrier; a plurality of polymerases; and a plurality of labeled nucleotides, each labeled nucleotide including a nucleotide; a 3′ OH blocking group attached to a sugar of the nucleotide; a dye label attached to a base of the nucleotide; and a plasmonic nanostructure attached to the base of the nucleotide; whereby at least one the polymerases incorporates an individual one of the labeled nucleotides into a nascent strand along one the template strands; andoptically imaging the incorporation.

It is to be understood that any features of the sixth aspect may be combined together in any desirable manner. Moreover, it is to be understood that any combination of features of the first aspect and/or of the second aspect and/or of the third aspect and/or of the fourth aspect and/or of the fifth aspect and/or of the sixth aspect may be used together, and/or may be combined with any of the examples disclosed herein to achieve the benefits as described in this disclosure, including, for example, enhanced fluorescence signals during sequencing imaging.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic illustration of an example of an incorporation mix;

FIG. 2A is a schematic illustration of an example plasmonic nanostructure attached to a polymerase through hybridized oligonucleotides;

FIG. 2B is a schematic illustration of an example plasmonic nanostructure attached to a polymerase through hybridized oligonucleotides and an oligonucleotide tether that is long enough to wrap around the plasmonic nanostructure;

FIG. 3 is a schematic illustration of an incorporation event involving the incorporation mix of FIG. 1;

FIG. 4 includes schematic illustrations depicting the chemical structures of different examples of labeled nucleotides;

FIG. 5A is a schematic illustration of another example of an incorporation mix;

FIG. 5B is a schematic illustration of another example of an incorporation mix;

FIG. 6 is a schematic illustration of an incorporation event involving the incorporation mix of FIG. 5A;

FIG. 7A through FIG. 7C are schematic illustrations of example kits including different examples of the incorporation mix and different examples of an imaging mix;

FIG. 8A is a top view of an example of a flow cell;

FIG. 8B is an enlarged, cross-sectional view, taken along the 8B-8B line of FIG. 8A, depicting an example of a flow channel and non-patterned sequencing surfaces of the flow cell; and

FIG. 8C is an enlarged, cross-sectional view, taken along the 8C-8C line of FIG. 8A, depicting of an example of a flow channel and patterned sequencing surfaces of the flow cell.

### DETAILED DESCRIPTION

In the examples disclosed herein, nucleotide base calling during sequencing is performed via the detection of plasmonic enhanced optical signals. Signal enhancement via plasmonic resonance is achieved through precise positioning of the plasmonic nanostructures with respect to dye labels of incorporated labeled nucleotides. The mechanisms and techniques disclosed herein localize the plasmonic nanostructure within signal enhancing proximity of the dye label, which is a component of the labeled nucleotide that has been incorporated into a nascent nucleic acid strand during sequencing. By “signal enhancing proximity,” it is meant that the plasmonic nanostructure and the dye label are separated by a distance which i) prevents quenching that can occur when the plasmonic nanostructure and the dye label are positioned too close to each other, and ii) increases plasmonic enhancement that can drop significantly at greater distances. The distance corresponding with signal enhancing proximity may range from greater than 0 nm to about 100 nm, but is dependent upon the plasmonic nanostructure (e.g., composition, shape, size) as well as the dye label. In some instances, the distance corresponding with signal enhancing proximity ranges from about 0.1 nm to about 25 nm, e.g., from about 1 nm to about 20 nm, etc. In one specific example, the distance corresponding with signal enhancing proximity ranges from about 3 nm to about 12 nm. The mechanisms and techniques disclosed herein allow the plasmonic nanostructure to be held within signal enhancing proximity of the dye label during imaging, and thus the plasmonic nanostructure is able to enhance the optical signal of the dye label.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, adjacent, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

An “acrylamide monomer” is a monomer with the structure

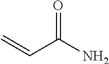

or a monomer including an acrylamide group. Examples of the monomer including an acrylamide group include azido acetamido pentyl acrylamide:

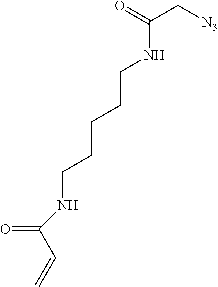 [SMILES: C=CC(=O)NCCCCCNC(=O)CN=[N+]=[N-]]

### and N-isopropylacrylamide:

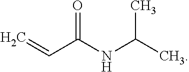

Other acrylamide monomers may be used.

An “aldehyde,” as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

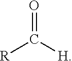

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen (e.g.,

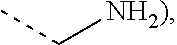

C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, the term “attached” refers to the state of two things being joined, fastened, adhered, connected or bound to each other, either directly or indirectly. For example, a nucleic acid can be attached to a polymeric hydrogel by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” as used herein refers to —COON.

The term “complex” refers to a polymerase that has a plasmonic nanostructure linked thereto.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

As used herein, the term “depression” refers to a discrete concave feature defined in a substrate and having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

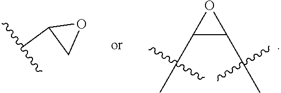

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be an area defined between two bonded components, which can selectively receive a liquid sample. In some examples, the flow channel may be defined between two substrates, and thus may be in fluid communication with the active areas of each of the substrates. In other examples, the flow channel may be defined between a substrate and a lid, and thus may be in fluid communication with active areas of the substrate.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

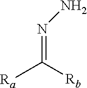

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a substrate that separates depressions or surrounds a lane. For example, an interstitial region can separate one depression of an array from another depression of the array. For another example, an interstitial region can separate one lane of a flow cell from another lane of a flow cell. The depressions and lanes that are separated from each other can be discrete, i.e., lacking physical contact with each other. In many examples, the interstitial region is continuous, whereas the depressions or lanes are discrete, for example, as is the case for a plurality of depressions or lanes defined in or on an otherwise continuous surface. The separation provided by an interstitial region can be partial or full separation. Interstitial regions may have a surface material that differs from the surface material of the depressions or lanes. For example, depressions or lanes can have the polymeric hydrogel and primers therein, and the interstitial regions can be free of both the polymeric hydrogel and primers.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

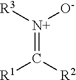

group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). A “labeled nucleotide” is a nucleotide that has at least a dye label attached thereto.

“Plasmonic nanostructures” include any independent structure capable of exhibiting plasmon resonance.

The term “polymeric hydrogel” refers to a semi-rigid polymer that is permeable to liquids and gases. The polymeric hydrogel can swell when liquid (e.g., water) is taken up and that can contract when liquid is removed, e.g., by drying. While a hydrogel may absorb water, it is not water-soluble.

As used herein, the term “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymeric hydrogel. The primer length can be any number of bases long and can include a variety of non-natural nucleotides. In an example, the sequencing primer is a short strand, ranging from 10 to 60 bases, or from 20 to 40 bases.

The phrase “substantially define the distance” is used to describe component(s) that create the desired separation between the plasmonic nanostructure and the dye label during imaging. As used herein, this phrase means that the listed component(s) generate at least 85% of the desired separation between the plasmonic nanostructure and the dye label. The other 15% may include a portion of the labeled nucleotide (e.g., the base), air, or fluid that may be present between the plasmonic nanostructure and the dye label. It is to be understood that the components are also flexible and able to rapidly move in solution. As such, the distance between the plasmonic nanostructure and the dye label may slightly change during imaging.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

Plasmonic Nanostructures

As noted herein, plasmonic nanostructures include any independent structure capable of exhibiting plasmon resonance. Plasmon resonance is the phenomenon where the electrons in the material surface layer are excited by photons of incident light with a certain angle of incidence, and then propagate parallel to the material surface. The surfaces of plasmonic nanostructures can strongly confine an electromagnetic field through its coupling to the propagating or localized surface plasmons. This interaction is associated with a large enhancement of the local electrical field, which in turn can enhance the excitation and emission rates and decrease the lifetimes of excited states of fluorescence emitters. This results in an amplified fluorescence signal and may also improve resistance to photobleaching.

Any material capable of plasmon resonance, referred to herein as a “plasmonic material”, may be used as the plasmonic nanostructure. Several metals (e.g., gold, silver, tin, rhodium, ruthenium, palladium, osmium, iridium, platinum, copper, aluminum, etc.), doped semi-metals (e.g., doped silicon), direct bandgap semiconductors (e.g., gallium arsenide), and metal composites are capable of plasmon resonance. Metal composites may include two or more of the metals listed above. As examples, a two-metal composite includes silver and gold and a three-metal composite includes silver, gold, and platinum. In any of the examples set forth herein, the plasmonic nanostructure may be selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

In an example, the plasmonic nanostructures are spherical nanoparticles. In another example, the plasmonic nanostructures are non-spherical nanoparticles, such as cubes, triangular, rod shaped, cage-like (e.g., non-spherical, hollow particles having a porous shell), etc. In still another example, the plasmonic nanostructures are irregularly shaped nanoparticles.

The plasmonic nanostructures may also be solid, hollow, or core-shell structures. The core-shell structure has one material as the core and another material as the shell at least partially encapsulating the core. In some examples, two different plasmonic materials are used as the core and shell. In other examples, the core is a plasmonic material and the shell is a non-plasmonic material. Some examples of suitable shell materials include silica, metal oxides, such as alumina, titania, and tantalum oxides, proteins, such as bovine serum albumin, and organic polymers that are transparent to the wavelengths used during sequencing, such as poly(methyl methacrylate) (PMMA), poly(lactic acid) (PLA), and poly(methyl acrylate) (PMA). The non-plasmonic material does not interfere with the plasmonic resonance of the core, and its thickness can be adjusted to help position the core within signal enhancing proximity of the dye label. As one example, when the silica shell of a core-shell plasmonic nanostructure is functionalized with a first member of a binding pair that can bind to a second member of a binding pair that is, e.g., attached to a labeled nucleotide, the thickness of the silica shell and the length of the bound members of the binding pair (including any linkers) substantially define the distance between the dye label and the plasmonic nanostructure core. As another example, when the silica shell of a core-shell plasmonic nanostructure is functionalized with a first member of a binding pair that can bind to a second member of a binding pair that is, e.g., attached to a polymerase, the thickness of the silica shell, the length of the bound members of the binding pair (including any linking molecules), and the size of the polymerase substantially define the distance between the dye label and the plasmonic nanostructure core.

The dimensions of the plasmonic nanostructure may vary depending upon its shape. In the examples disclosed herein, the largest dimension (e.g., diameter, length, median, etc.) of the plasmonic nanostructure is on the nanoscale, and thus ranges from about 1 nm to less than 1000 nm. In some examples, the nanostructures are nanoparticles having a diameter of greater than or equal to 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, or greater than or equal to 100 nm.

The plasmonic enhancement of optical signals is strongly dependent upon the distance of the plasmonic nanostructure from the dye label whose signals are to be enhanced. The mechanisms and techniques disclosed herein allow the plasmonic nanostructure to be held within signal enhancing proximity of the dye label during imaging. Each of these mechanisms and techniques will now be described.

Plasmonic Nanostructure in an Incorporation Mix

The plasmonic nanostructures may be included in an incorporation mix. In some examples, the plasmonic nanostructures are part of a complex that also includes a polymerase. In these examples, the complex is the mechanism by which the plasmonic nanostructure is held within signal enhancing proximity of the dye label. In other examples, the nanostructures are part of a labeled nucleotide. In these examples, the labeled nucleotide is the mechanism by which the plasmonic nanostructure is held within signal enhancing proximity of the dye label.

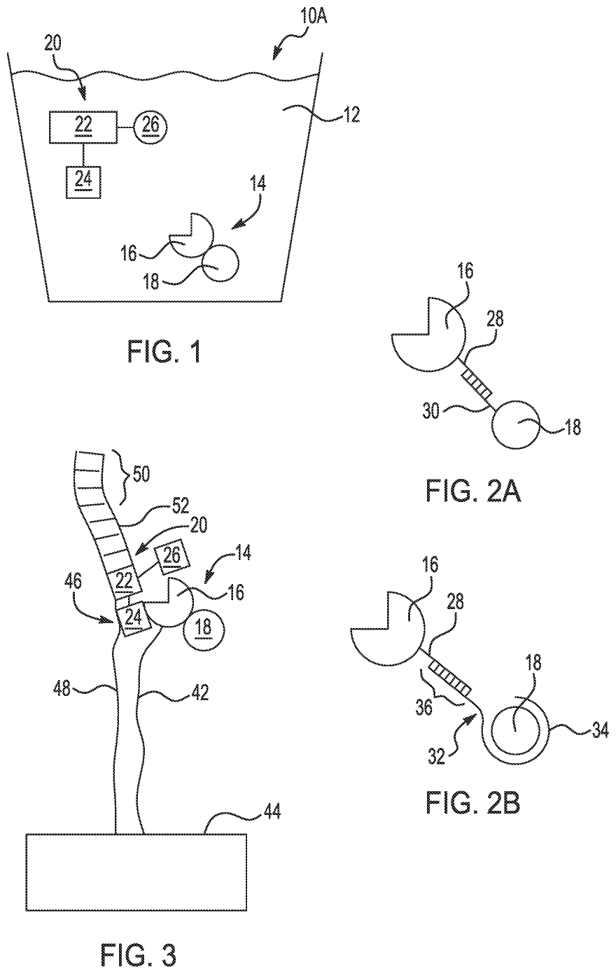

One example of the incorporation mix is shown schematically in FIG. 1. This example incorporation mix 10A includes a liquid carrier 12, a complex 14 including a polymerase 16 and a plasmonic nanostructure 18 linked to the polymerase 16, and a labeled nucleotide 20 including a nucleotide 22, a 3′ OH blocking group 24 attached to a sugar of the nucleotide 22, and a dye label 26 attached to a base of the nucleotide 22.

The liquid carrier 12 of the incorporation mix 10A may be water and/or an ionic salt buffer fluid, such as saline citrate at milli-molar to molar concentrations, sodium chloride, potassium chloride, phosphate buffered saline, etc., and/or other buffers, such as tris(hydroxymethyl)aminomethane (TRIS) or (4-(2-hydroxyethyl)-1-piperazineethanesulfonic acid) (HEPES). The liquid carrier 12 may also include catalytic metal(s) intended for the incorporation reaction, such as Mg²⁺, Mn²⁺, Ca²⁺, etc. A single catalytic metal or a combination of catalytic metals may be used, and the total concentration may range from about 0.01 mM to about 100 mM.

The complex 14 includes the polymerase 16 and the plasmonic nanostructure 18 linked to the polymerase 16. The concentration of the complex 14 in the incorporation mix 10A may range from about 1 nM to about 10 mM. As other examples, the concentration of the complex 14 in the incorporation mix 10A may range from about 50 nM to about 100 μM, from about 1 nM to about 50 μM, etc.

Any polymerase 16 that can accept the labeled nucleotides 22, and that can successfully incorporate the nucleotide base into a nascent strand along a template strand may be used. Examples polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol p, DNA Pol 13, DNA Pol a, and many others.

Because the plasmonic nanostructure 18 is linked to the polymerase 16 that performs labeled nucleotide incorporation, the polymerase 16 at least partially defines the space between the dye label 26 and the plasmonic nanostructure 18. As such, the size of the polymerase 16 may be selected to create the signal enhancing proximity. In one example, the size of the polymerase 16 ranges from about 3 nm to about 9 nm. In another example, the size of the polymerase 16 ranges from about 4 nm to about 7 nm. In still another example, the size of the polymerase 16 is about 5 nm.

In addition to the polymerase 16, the complex 14 also includes the plasmonic nanostructure 18 linked to the polymerase 16. The plasmonic nanostructure 18 of the complex 14 may be any of the examples set forth herein. The polymerase 16 has an active site for nucleotide incorporation, and it may be desirable to link the plasmonic nanostructure 18 to a region of the polymerase 16 that is away from the active site. For example, internal or terminal tags that are distal from the active site may be desirable for plasmonic nanostructure 18 attachment so that the polymerase 16 activity is not inhibited. Any of the following techniques may be used to link the plasmonic nanostructure 18 to the polymerase 16.

In one example, the plasmonic nanostructure 18 is chemically conjugated to the polymerase 16. Chemical conjugation may be achieved with a binding pair, where the plasmonic nanostructure 18 is functionalized with a first member of the binding pair and the polymerase 16 includes or is functionalized with a second member of the binding pair. In example binding pairs, the first member and the second member respectively include a NiNTA (nickel-nitrilotriacetic acid) ligand and a histidine tag, or streptavidin or avidin and biotin, or a spytag and a spycatcher, or maleimide and cysteine, or azide and dibenzocyclooctyne (DBCO). Some of these examples involve chemical conjugation to amines or cysteines present on the polymerase 16. Naturally occurring or engineered cysteines on the polymerase 16 readily react with the surface of some functionalized plasmonic nanostructures (e.g., gold, silver, platinum, alloys thereof). Naturally occurring or engineered cysteines on the polymerase 16 also readily react with maleimide groups that are incorporated on the surface of some functionalized plasmonic nanostructures. Any plasmonic nanoparticle can be functionalized with carboxyl groups, which can be reacted with naturally occurring or engineered amines on the polymerase 16 by activating them with EDC (1-Ethyl-3-[3-dimethylaminopropyl]carbodiimide hydrochloride). N-hydroxysuccinimide esters (NHS esters) may be used to label amine groups on the polymerase 16, such as lysine. In an example, N-Hydroxysuccinimide (NHS) chemistry may be employed at pH ranges where only the N-terminal amines are reactive (for instance, pH 7), such that a single plasmonic nanostructure 18 is added per polymerase 16. Chemical conjugation may also be achieved through a π-clamp mediated cysteine conjugation, a SUMO (small ubiquitin-like modifier) protein, or any other suitable conjugation method.

In another example, the plasmonic nanostructure 18 is attached to the polymerase 16 through hybridized oligonucleotides. This example is shown schematically in FIG. 2A. In this example, a single stranded oligonucleotide 28 is attached to the polymerase 16, and its complementary single stranded oligonucleotide tether 30 is attached to the plasmonic nanostructure 18. The oligonucleotide 28 hybridizes to the complementary oligonucleotide tether 30 to link the plasmonic nanostructure 18 to the polymerase 16. Any of the chemical conjugation techniques disclosed herein may be used to attach the oligonucleotide 28 to the polymerase 16 and to attach the complementary oligonucleotide tether 30 to the plasmonic nanostructure 18. As two examples, a thiolated oligonucleotide can bond directly to the surface of a gold nanoparticle or a silver nanoparticle. Additionally, the complementary oligonucleotide tether 30 may be attached to the plasmonic nanostructure 18 through a variety of other chemistries, including an azide-alkyne click reaction, a copper-free click reaction, an aldehyde functional oligo-amine plasmonic nanostructure reaction.

The oligonucleotide 28 and the complementary oligonucleotide tether 30 have the same length, which may range from about 5 nucleotides to about 30 nucleotides.

In this example, the polymerase 16 and the hybridized oligonucleotides 28 and 30 at least partially define the distance between the dye label 26 and the plasmonic nanostructure 18. As such, the size of the polymerase 16 and the length of the oligonucleotides 28 and 30 may be selected so that during imaging, the plasmonic nanostructure 18 is brought within signal enhancing proximity of the dye label 26.

In yet another example, the plasmonic nanostructure 18 is attached to the polymerase 16 through an oligonucleotide tether 32 that wraps around the plasmonic nanostructure 18. This oligonucleotide tether 32 includes a portion 34 that is long enough to wrap around the plasmonic nanostructure 18 and a portion 36 that is complementary to an oligonucleotide 28 that is attached to the polymerase 16. The length of the portion 34 that is long enough to wrap around the plasmonic nanostructure 18 will depend upon the size of the plasmonic nanostructure 18. In an example, the plasmonic nanostructure 18 has a diameter ranging from about 5 nm to about 12 nm, and the portion 34 that is long enough to wrap around the plasmonic nanostructure 18 may include from about 40 nucleotides to about 150 nucleotides. These nucleotides can selectively bind with the surface of the plasmonic nanostructure 18. In one example, the plasmonic nanostructure 18 is gold and the portion 34 that is long enough to wrap around the plasmonic nanostructure 18 is poly-adenine.

The complementary portion 36 of the oligonucleotide tether 32 hybridizes to the oligonucleotide 28 on the polymerase 16 to link the plasmonic nanostructure 18, which is wrapped in the portion 34, to the polymerase 16. In this example, the complementary portion 36 and the oligonucleotide 28 have the same length, which may range from about 5 nucleotides to about 20 nucleotides. Any of the chemical conjugation techniques disclosed herein may be used to attach the oligonucleotide 28 to the polymerase 16.

In this example, the polymerase 16 and the hybridized oligonucleotide 28 and portion 36 at least partially define the distance between the dye label 26 and the plasmonic nanostructure 18. As such, the size of the polymerase 16 and the length of the oligonucleotide 28 and the portion 36 may be selected so that during imaging, the plasmonic nanostructure 18 is brought within signal enhancing proximity of the dye label 26.

In some examples, the complex 14 may also include an additional component to improve retention of the complex 14 in the flow cell after a wash cycle.

In one example to improve complex retention after the wash cycle, the complex 14 includes DNA binding domains genetically fused to the polymerase 16. In one example, the DNA binding domains may include single stranded DNA (ss-DNA) binding domains, which can attach, e.g., to an adapter portion of the template strand in the flow cell. The ss-DNA binding domains may be non-sequence specific. Examples of these DNA binding domains include oligonucleotide/oligosaccharide/oligopeptide-binding (OB) folds, K homology (KH) domains, RNA recognition motifs (RRMs), and whirly domains. In another example, the DNA binding domains may include double stranded DNA (ds-DNA) binding domains, which can attach, e.g., the double stranded portion of the sequencing primer/template duplex. Examples of these DNA binding domains include TOPO-V helix-hairpin-helix domains and PCNA (proliferating cell nuclear antigen).

In another example to improve complex retention after the wash cycle, the complex 14 may include a surface tether 42 (shown in FIG. 3). The surface tether 42 is used to anchor the complex 14 to a patterned or non-patterned flow cell surface 44 during incorporation and imaging. In an example, the polymerase 16 includes the surface tether 42 attached to a surface thereof, and a flow cell surface binding agent is attached to the surface tether 42 (e.g., at an end opposite where the polymerase 16 is attached). In this example, the flow cell surface 44 is functionalized to attach the polymerase-bound surface tether 42 when the polymerase 16 is introduced into the flow cell. In another example, the flow cell includes the surface tether 42 attached to a surface 44 thereof, and a polymerase binding agent is attached to the surface tether 42. In this example, the polymerase surface is functionalized to attach to the flow cell-bound surface tether 42 when the polymerase 16 is introduced into the flow cell. In either of these examples, the surface tether 42 may include one member of a binding pair while the polymerase 16 carries the other member: biotin/avidin, maltose-binding protein/maltose, His6/NiNTA, Spycatcher002/Spytag002, or glutathione S-transferase/glutathione.

One specific example of a suitable surface tether 42 includes biotin-polyethylene glycol (PEG)-biotin. In this example, the biotin ends of the surface tether 42 are able to respectively conjugate to streptavidin or avidin located on the surface of the polymerase 16 and on the flow cell surface 44. As such, in this example, the biotin at one end is the flow cell surface binding agent. Another specific example of a suitable surface tether 42 includes an avidin protein on the flow cell surface 44. In this example, the polymerase 16 may be biotinylated. Still another specific example of a suitable surface tether 42 includes halogen-polyethylene glycol (PEG). In this example, PEG may be attached to the flow cell surface 44, and the halogen can attached to a HaloTag® attached to the polymerase 16.

The length of the surface tether 42 is sufficient to enable the surface bound polymerase 16 to bind anywhere at the junction 46 between a template strand 48 and a sequencing primer 50 or a growing nascent strand 52 extending from the sequencing primer 50. In an example, the surface tether 42 may have a length ranging from about 2 nm to about 200 nm, or from about 5 nm to about 150 nm, or from about 10 nm to about 100 nm, depending, in part upon the length of the library fragments to be sequenced on the flow cell surface 44.

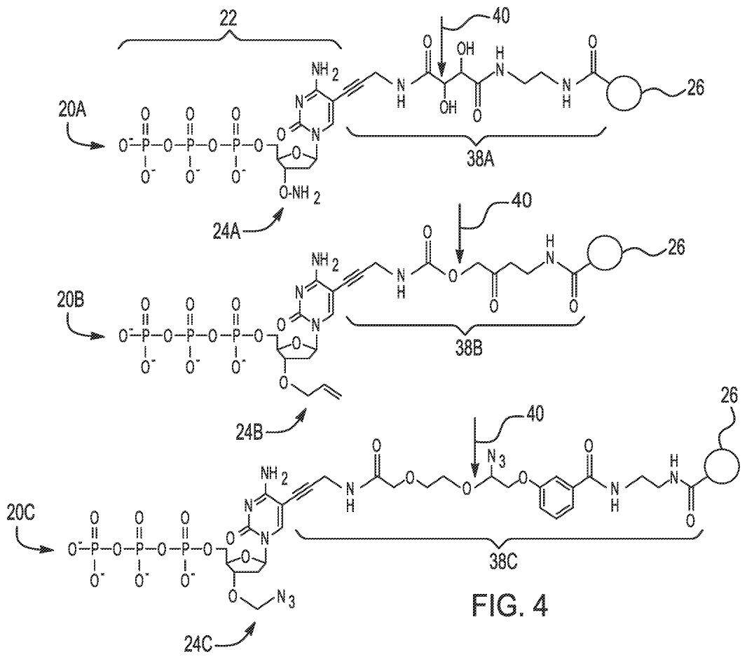

Referring back to FIG. 1, in addition to the complex 14, the incorporation mix 10A shown in FIG. 1 also includes the labeled nucleotide 20. As mentioned herein, the labeled nucleotide 20 includes the nucleotide 22, the 3′ OH blocking group 24 attached to the sugar of the nucleotide 22, and the dye label 26 attached to the base of the nucleotide 22. Several examples of the labeled nucleotide 20A, 20B, 20C are shown in FIG. 4.

The nucleotide 22 of the labeled nucleotide 20 includes a nitrogen containing heterocyclic base, a sugar, and one or more phosphate groups. Nucleotides 22 are monomeric units of a nucleic acid sequence. In RNA, the sugar is a ribose, and in DNA, the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of ribose or deoxyribose is bonded to N-1 of a pyrimidine or N-9 of a purine. The nucleotide 22 may be a monophosphate, or a polyphosphate form including several phosphate groups (e.g., tri-phosphate (i.e., gamma phosphate), tetra-phosphate, penta-phosphate, hexa-phosphate, etc.). A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). In FIG. 4, the base of the nucleotide 22 is cytosine, the sugar is deoxyribose, and the phosphate is a tri- or gamma-phosphate.

The nucleotide 22 of the labeled nucleotide 20 also includes a 3′ OH blocking group 24 attached thereto. The 3′ OH blocking group 24 may be linked to the 3′ oxygen atom of the sugar molecule in the nucleotide 22. The 3′ OH blocking group 24 may be a reversible terminator that allows only a single-base incorporation to occur in each sequencing cycle. The reversible terminator stops additional bases from being incorporated into a nascent strand 52 (FIG. 3) that is complementary to the template polynucleotide chain (also referred to herein as a template strand 48 (FIG. 3)). This enables the detection and identification of a single incorporated base. The 3′ OH blocking group 24 can subsequently be removed, enabling additional sequencing cycles to take place at each template polynucleotide chain. Examples of different 3′ OH blocking groups 24 are shown in FIG. 4 at reference numerals 24A, 24B, 24C, including a 3′-ONH₂ reversible terminator (shown at 24A), a 3′-O-allyl reversible terminator (i.e., —CH═CHCH₂, shown at 24B), and 3′-O-azidomethyl reversible terminator (i.e., —CH₂N₃, shown at 24C). Other suitable reversible terminators include o-nitrobenzyl ethers, alkyl o-nitrobenzyl carbonate, ester moieties, other allyl-moieties, acetals (e.g., tert-butoxy-ethoxy), MOM (—CH₂OCH₃) moieties, 2,4-dinitrobenzene sulfenyl, tetrahydrofuranyl ether, 3′ phosphate, ethers, —F, —H₂, —OCH₃, —N₃, —HCOCH₃, and 2-nitrobenzene carbonate.

The nucleotide 22 of the labeled nucleotide 20 also includes a dye label 26 attached to the base of the nucleotide 22. The dye label 26 may be any optically detectable moiety, including luminescent, chemiluminescent, fluorescent, fluorogenic, chromophoric and/or chromogenic moieties. Some examples of suitable optically detectable moieties include fluorescein labels, rhodamine labels, cyanine labels (e.g., Cy3, Cy5, and the like), and the ALEXA FLUOR® family of fluorescent dyes (from Molecular Probes, Inc.) and other fluorescent and fluorogenic dyes).

The dye label 26 may be attached to the base of the nucleotide 22 using any suitable linking molecule. In an example, the linking molecule is a spacer group of formula —((CH₂)₂O)ₙ— wherein n is an integer between 2 and 50. Other examples of the linking molecule are shown at reference numerals 38A, 38B, and 38C in FIG. 4. The linking molecule 38A, 38B, 38C includes a cleavage site 40, identified by the arrows in FIG. 4.

In one example, the incorporation mix 10A includes a mixture of different labeled nucleotides 20, which include different bases, e.g., A, T, G, C (as well as U or I). It may also be desirable to utilize a different type of dye label 26 for the different labeled nucleotides 20. For example, the fluorescent or fluorogenic labels may be selected so that each label absorbs excitation radiation and/or emits fluorescence, at a distinguishable wavelength from the other labels. Such distinguishable analogs provide an ability to monitor the presence of different labels 26 simultaneously in the same reaction mixture. In some examples, one of the four labeled nucleotides 20 in the incorporation mix 10A may include no dye label 26, while the other three labeled nucleotides 20 include different dye label 26.

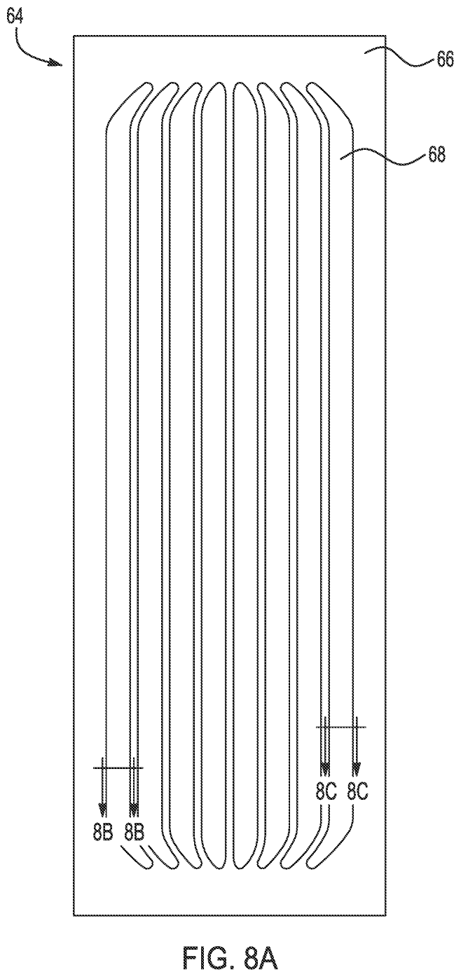

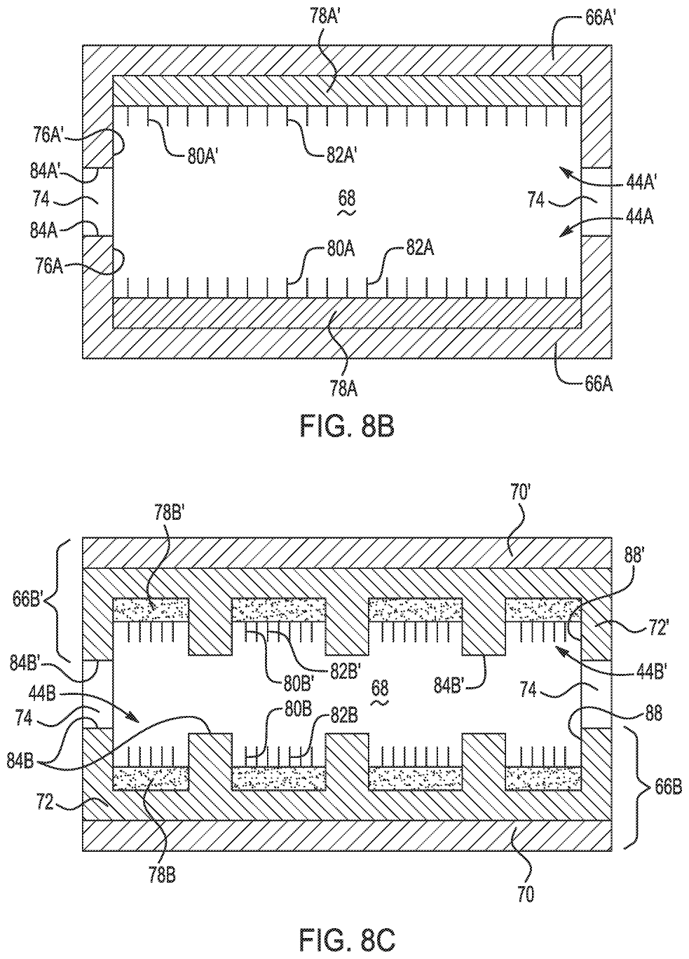

Referring back to FIG. 3, the following description relates to an example method that involves the incorporation mix 10A. Throughout this description, a flow cell is referenced and briefly described. Examples of the flow cell are shown in shown in FIG. 8A, FIG. 8B and FIG. 8C, and will be described in more detail below in reference to those figures.

During sequencing, a template strand 48 that is to be sequenced may be formed on the flow cell surface 44 using amplification primers (not shown in FIG. 3) that are immobilized on the flow cell surface 44. At the outset of template strand formation, library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the amplification primers on the flow cell surface 44. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to the flow cell. Multiple library templates are hybridized, for example, to one of two types of amplification primers immobilized on the flow cell surface 44.

Cluster generation may then be performed. In one example of cluster generation, the library templates are copied from the hybridized primers by 3′ extension using a high-fidelity DNA polymerase. The original library templates are denatured, leaving the copies immobilized on the flow cell surface 44. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters. Each cluster of double stranded bridges is denatured. In an example, the reverse strand is removed by specific base cleavage, leaving forward template strands. While a single template strand 48 is shown in FIG. 3, clustering results in the formation of several template strands 48 immobilized on the flow cell surface 44 (e.g., across the lane as shown in FIG. 8B or in depressions as shown in FIG. 8C). This example of clustering is referred to as bridge amplification, and is one example of the amplification that may be performed. It is to be understood that other amplification techniques may be used, such as the exclusion amplification (Examp) workflow (IIlumina Inc.).

A sequencing primer 50 may be introduced that hybridizes to a complementary portion of the sequence of the template strand 48. This sequencing primer 50 renders the template strand 48 ready for sequencing using the incorporation mix 10A.

An example sequencing method involves introducing the incorporation mix 10A to the flow cell including clusters of template strands 48 (with the sequencing primer 50 hybridized thereto), the incorporation mix 10A including a plurality of the complexes 14 and a plurality of labeled nucleotides 20, whereby at least one the polymerases 16 i) incorporates an individual one of the labeled nucleotides 20 into a nascent strand 52 along one the template strands 48, and ii) maintains its linked plasmonic nanostructure 18 within proximity of the individual one of the labeled nucleotides 20; and optically imaging the incorporation while the plasmonic nanostructure 18 is maintained.

In this example method, any example of the incorporation mix 10A is introduced into the flow cell, e.g., via an input port. When the incorporation mix 10A is introduced into the flow cell, the mix 10A enters a flow channel, and contacts the surface(s) 44 where the template strands 48 are present.

The incorporation mix 10A is allowed to incubate in the flow cell, and labeled nucleotides 20 are incorporated by respective polymerases 16 of the complexes 14. As shown in FIG. 3, during incorporation, one of the labeled nucleotides 20 is incorporated, by a respective polymerase 16, into one nascent strand 52 that extends one sequencing primer 50 and that is complementary to one of the template strands 48. Incorporation is performed in a template strand dependent fashion, and thus detection of the order and type of labeled nucleotides 20 added to the nascent strand 52 can be used to determine the sequence of the template strand 48. Incorporation occurs in at least some of the template strands 48 across the flow cell surface 44 during a single sequencing cycle. As such, in at least some of the template strands 48 across the flow cell, respective polymerases 16 extend the hybridized sequencing primer 50 by one of the labeled nucleotides 20 in the incorporation mix 10A.

The incorporated labeled nucleotides 20 include the reversible termination property due to the presence of the 3′ OH blocking group 24, which terminates further sequencing primer extension once the labeled nucleotide 20 has been added.

After a desired time for incubation and incorporation, the incorporation mix 10A, including non-incorporated labeled nucleotides 20, may be removed from the flow cell during a wash cycle. The wash cycle may involve a flow-through technique, where a washing solution (e.g., buffer) is directed into, through, and then out of flow channel, e.g., by a pump or other suitable mechanism.

At least some of the polymerases 16 that perform incorporation, and their respective complexes 14, remain in place at the junction 46 between the template strand 48 and the most recently incorporated labeled nucleotide 20 after the wash cycle. In one example, from about 20% to about 90% of the complexes 14 remain in the flow cell after the wash cycle. While one example range is provided, it is to be understood that the percentage of remaining complexes 14 may be more or less, depending on a variety of different factors. As mentioned herein, to increase the percentage of complexes 14 retained during and after the wash cycle, the complexes 14 may be tethered to the template strand 48 (e.g., via the DNA binding domain) or to the flow cell surface 44 (e.g., via the surface tether 42). Because each retained complex 14 includes a plasmonic nanostructure 18, these plasmonic nanostructures 18 are also retained in place at the junction 46 between the template strand 48 and the most recently incorporated labeled nucleotide 20, as shown in FIG. 3. As such, the plasmonic nanostructure 18 is held within signal enhancing proximity of the dye label 26 after incorporation.

Without further incorporation taking place, the most recently incorporated labeled nucleotides 20 can be detected through an imaging event. During the imaging event, an illumination system (not shown) may provide an excitation light to the flow cell surface 44. The dye labels 26 of the incorporated labeled nucleotides 20 emit optical signals in response to the excitation light. Additionally, because the retained plasmonic nanostructures 18 are held within signal enhancing proximity of the respective dye labels 26, the signals from the dye labels 26 can be enhanced through plasmonic resonance.

After imaging is performed, a cleavage mix may then be introduced into the flow cell. In this example, the cleavage mix is capable of i) removing the 3′ OH blocking group 24 from the incorporated nucleotides 20, and ii) cleaving the dye label 26 from the incorporated nucleotides 20. Examples of 3′ OH blocking groups 24 and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable dye label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

The wash with the cleavage mix, or a subsequent wash may also remove the retained complexes 14 that are not otherwise linked to the template strand 48 or the flow cell surface 44.

When the complexes 14 are linked to the template strand 48 (e.g., via the DNA binding domain) or to the flow cell surface 44 (e.g., via the surface tether 42), an additional complex-removing agent may be added to the cleavage mix. The complex-removing agent will depend upon the linkage formed between the complex 14 and the template strand 48 or between the complex 14 and the flow cell surface 44. Some of the example dye label cleaving agents set forth herein may be able to cleave the complexes 14. In one example, a biotin-streptavidin binding pair links the surface tether 42 to the flow cell surface 44. In this example, the complex-removing agent may be sodium dodecyl sulfate (SDS), a combination of urea and biotin, or a combination of from about 10% by volume to about 50% by volume of a formamide reagent including formamide and an optional buffer and a balance of a salt buffer including sodium chloride, sodium citrate, and a biocompatible surfactant. In another example, an excess of one of the binding pair components may be capable of breaking the bond between the bind pair (e.g., between the polymerase 16 and the surface tether 42 or between the surface tether 42 and the flow cell surface 44), either of which leaves a binding site open on the flow cell surface 44. In still another example, a nuclease or allyl cleavage chemistry may be added to cleave the surface tether 42. In yet another example, exogenous DNA may be added to encourage the DNA-binding domain to release from the surface conjugated DNA.

Wash(es) may take place between the various fluid delivery steps. The sequencing cycle can then be repeated “n” times to extend the sequencing primer 50 by n nucleotides, thereby detecting a sequence of length n. In some examples, paired-end sequencing may be used, where the forward strands are sequenced and removed, and then reverse strands are constructed and sequenced.

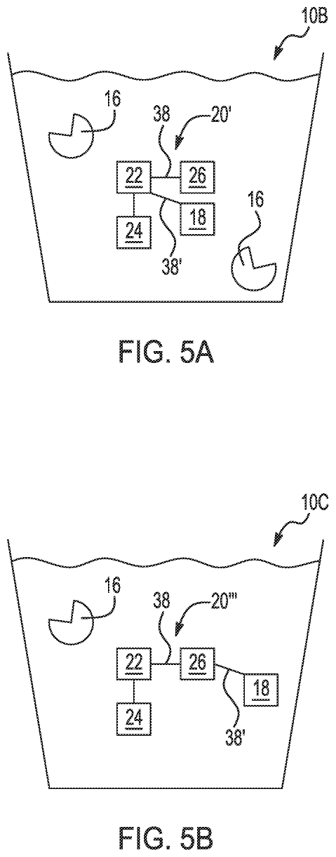

Referring now to FIG. 5A, another example of the incorporation mix 10B is schematically depicted. This example incorporation mix 10B includes another example of the labeled nucleotide 20′. This labeled nucleotide 20′ includes a nucleotide 22, a 3′ OH blocking group 24 attached to a sugar of the nucleotide 22, a dye label 26 attached to a base of the nucleotide 22, and a plasmonic nanostructure 18 attached to the base of the nucleotide 22.

Any of the nucleotides 22, 3′ OH blocking groups 24, and dye labels 26 described herein may be used for the labeled nucleotide 20′. Additionally, any of the plasmonic nanostructures 18 described herein may also be used for the labeled nucleotide 20′.

In this example, a first linking molecule 38 attaches the dye label 26 to the base of the nucleotide 22 and a second linking molecule 38′ attaches the plasmonic nanostructure 18 to the base of the nucleotide 22. The lengths of the first and second linking molecules 38, 38′ may depend upon how the linking molecules 38, 38′ are attached to the base. In some examples, the lengths will range from about 1 nm to about 12 nm, or up to about 100 nm when the distance associated with signal enhancing proximity is between 0.1 nm and 100 nm.

The first and/or second linking molecules 38, 38′ may be any of the linking molecules described for linking the dye label 26 to the base, such as the spacer group —((CH₂)₂O)ₙ— (n ranges from 2 to 50) or those examples shown at reference numerals 38A, 38B, and 38C in FIG. 4. In some examples, the first and/or second linking molecules 38, 38′ are selected to be the same type of linker so that they may be cleaved using the same mechanism after incorporation and imaging. Another example of the second linking molecule 38′ is ds-DNA (double stranded DNA). The rigidity of the ds-DNA linker may be particularly desirable. In any of these examples, the second linking molecule 38′ may include an end group that can chemically conjugate to the plasmonic nanostructure 18. As one example, biotin may be added to the end of the second linking molecule 38′, and the plasmonic nanostructures 18 may be functionalized with avidin or streptavidin that can bind to the biotin. As another example, thiol may be added to the end of the second linking molecule 38′, which can bond directly to the surface of a gold nanoparticle.

As shown in FIG. 5A, this example of the incorporation mix 10B also includes a liquid carrier 12 and a polymerase 16. The concentration of the polymerase 16 and/or the labeled nucleotide 20′ may range from about 50 nM to about 100 μM, from about 1 nM to about 50 μM, etc.

The liquid carrier 12 of the incorporation mix 10B may be water and/or any of the buffers set forth herein.

The polymerase 16 of the incorporation mix 10B may be any of the examples provided herein. In these examples, it is to be understood that the polymerase 16 in the incorporation mix 10B is not part of the complex 14. As such, the polymerase 16 is not linked to the plasmonic nanostructure 18. Because the polymerase 16 and plasmonic nanostructure 18 are separate, the polymerase 16 in these examples may not be functionalized for tethering to the template strand 48 (e.g., via the DNA binding domain) or to the flow cell surface 44 (e.g., via the surface tether 42).

Referring now to FIG. 5B, another example of the incorporation mix 10C is schematically depicted. This example incorporation mix 10C includes another example of the labeled nucleotide 20′″. This labeled nucleotide 20′″ includes a nucleotide 22, a 3′ OH blocking group 24 attached to a sugar of the nucleotide 22, a dye label 26 attached to a base of the nucleotide 22, and a plasmonic nanostructure 18 attached to the dye label 26. The attachment of the plasmonic nanoparticle 18 to the dye label 26 may be less sterically congested than the example labeled nucleotide 20′ shown in FIG. 5A.

Any of the nucleotides 22, 3′ OH blocking groups 24, and dye labels 26 described herein may be used for the labeled nucleotide 20′″. Additionally, any of the plasmonic nanostructures 18 described herein may also be used for the labeled nucleotide 20′″.

In this example, the first linking molecule 38 attaches the dye label 26 to the base of the nucleotide 22 and the second linking molecule 38′ attaches the plasmonic nanostructure 18 to the dye label 26. The total length of the first and second linking molecules 38, 38′ may depend upon how the linking molecule 38 is attached to the base and how the linking molecule 38′ is attached to the dye label 26. In some examples, the length of the linking molecule 38′ ranges from about 1 nm to about 12 nm, or up to about 100 nm when the distance associated with signal enhancing proximity is between 0.1 nm and 100 nm.

The first and/or second linking molecules 38, 38′ may be any of the linking molecules described for linking the dye label 26 to the base, such as the spacer group —((CH₂)₂O)ₙ— (n ranges from 2 to 50) or those examples shown at reference numerals 38A, 38B, and 38C in FIG. 4.

As shown in FIG. 5B, this example of the incorporation mix 10C also includes a liquid carrier 12 and a polymerase 16. The concentration of the polymerase 16 and/or the labeled nucleotide 20′″ may range from about 50 nM to about 100 μM, from about 1 nM to about 50 μM, etc.

The liquid carrier 12 of the incorporation mix 10C may be water and/or any of the buffers set forth herein. The liquid carrier 12 of the incorporation mix 10C may also include catalytic metal(s).

The polymerase 16 of the incorporation mix 10C may be any of the examples provided herein. In these examples, it is to be understood that the polymerase 16 in the incorporation mix 10C is not part of the complex 14. As such, the polymerase 16 is not linked to the plasmonic nanostructure 18. Because the polymerase 16 and plasmonic nanostructure 18 are separate, the polymerase 16 in these examples may not be functionalized for tethering to the template strand 48 (e.g., via the DNA binding domain) or to the flow cell surface 44 (e.g., via the surface tether 42).

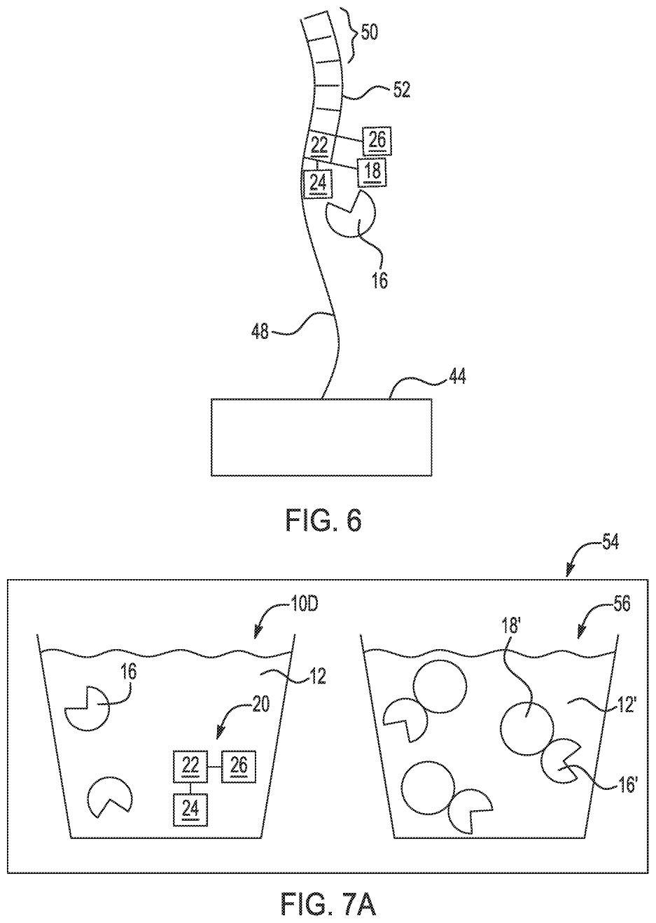

Referring now to FIG. 6, the following description relates to an example method that involves the incorporation mix 10B. This method also utilizes examples of the flow cell shown and described in detail in reference to FIG. 8A, FIG. 8B and FIG. 8C. At the outset of this method, the template strands 48 have been formed as described in reference to FIG. 3.

As shown in FIG. 6, the sequencing primer 50 may be introduced that hybridizes to a complementary portion of the sequence of the template strand 48. This sequencing primer 50 renders the template strand 48 ready for sequencing using the incorporation mix 10B.

An example sequencing method involves introducing the incorporation mix 10B to the flow cell including clusters of template strands 48 (with the sequencing primer 50 hybridized thereto), the incorporation mix 10B including a liquid carrier 12, a plurality of polymerases 16, and a plurality of labeled nucleotides 20′, whereby at least one the polymerases 16 incorporates an individual one of the labeled nucleotides 20′ into a nascent strand 52 along one the template strands 48; and optically imaging the incorporation.

In this example method, any example of the incorporation mix 10B is introduced into the flow cell, e.g., via an input port. When the incorporation mix 10B is introduced into the flow cell, the mix 10B enters a flow channel, and contacts the surface(s) 44 where the template strands 48 are present.

The incorporation mix 10B is allowed to incubate in the flow cell, and labeled nucleotides 20′ are incorporated by respective polymerases 16. As shown in FIG. 6, during incorporation, one of the labeled nucleotides 20′ is incorporated, by a respective polymerase 16, into one nascent strand 52 that extends one sequencing primer 50 and that is complementary to one of the template strands 48. This incorporation is also performed in a template strand dependent fashion, and thus detection of the order and type of labeled nucleotides 20′ added to the nascent strand 52 can be used to determine the sequence of the template strand 48. Incorporation occurs in at least some of the template strands 48 across the flow cell surface 44 during a single sequencing cycle.

The incorporated labeled nucleotides 20′ include the reversible termination property due to the presence of the 3′ OH blocking group 24, which terminates further sequencing primer extension once the labeled nucleotide 20′ has been added.

After a desired time for incubation and incorporation, the incorporation mix 10B, including non-incorporated labeled nucleotides 20′, may be removed from the flow cell during a wash cycle. The wash cycle may involve a flow-through technique, where a washing solution (e.g., buffer) is directed into, through, and then out of flow channel, e.g., by a pump or other suitable mechanism.

Because respective plasmonic nanostructures 18 are linked to the incorporated labeled nucleotides 20′, these plasmonic nanostructures 18 are retained in place within signal enhancing proximity of the dye label 26 after incorporation.

Without further incorporation taking place, the most recently incorporated labeled nucleotides 20′ can be detected through an imaging event. During the imaging event, an illumination system (not shown) may provide an excitation light to the flow cell surface 44. The dye labels 26 of the incorporated labeled nucleotides 20′ emit optical signals in response to the excitation light, and the respective plasmonic nanostructures 18 of the incorporated labeled nucleotides 20′ enhance these optical signals through plasmonic resonance.

After imaging is performed, a cleavage mix may then be introduced into the flow cell. In this example, the cleavage mix is capable of i) removing the 3′ OH blocking group 24 from the incorporated nucleotides 20′, ii) cleaving the dye label 26 from the incorporated nucleotides 20′, and iii) cleaving the plasmonic nanostructure 18 from the incorporated nucleotides 20′.

In this example, any of the de-blocking agents/components described herein for cleaving the 3′ OH blocking group 24 may be used. Also in this example, any of the cleaving agents/components described herein for cleaving the dye label 26 may be used. When the linking molecules 38, 38′ used to respectively attach the dye label 26 and the plasmonic nanostructure 18 are the same, the cleavage mix may include a single cleaving agent/component that can cleave the linking molecules 38, 38′. When the linking molecules 38, 38′ used to respectively attach the dye label 26 and the plasmonic nanostructure 18 are different, the cleavage mix may include different cleaving agents/components that can cleave the respective linking molecules 38, 38′. As one example, restriction enzymes targeting specific sequences of each linking molecule 38, 38′ may be used.

Wash(es) may take place between the various fluid delivery steps. The sequencing cycle can then be repeated n times to extend the sequencing primer 50 by n nucleotides, thereby detecting a sequence of length n. In some examples, paired-end sequencing may be used, where the forward strands are sequenced and removed, and then reverse strands are constructed and sequenced.

The incorporation mix 10C may be used in the method described in reference to FIG. 6 instead of the incorporation mix 10B. The method (e.g., incorporation, incubation, signal enhancement, etc.) will be similar to that described in reference to FIG. 6, except the cleavage process may be simplified. In the incorporation mix 10C, the plasmonic nanostructure 18 is attached to the dye label 26. Thus, when the dye label 26 is cleaved, the plasmonic nanostructure 18 will also be cleaved. As such, an additional cleaving agent for the linking molecule 38′ is not used during the method.

Plasmonic Nanostructure in an Imaging Mix

In other examples disclosed herein, the plasmonic nanostructures 18 may be included in an imaging mix rather than in the incorporation mix 10A or 10B or 10C. In these examples, the nanostructures 18 are functionalized so that they can associate with an incorporated labeled nucleotide (e.g., labeled nucleotide 20). In these examples, the functionalization is at least part of the mechanism by which the plasmonic nanostructure 18 is localized within signal enhancing proximity of the dye label 26.

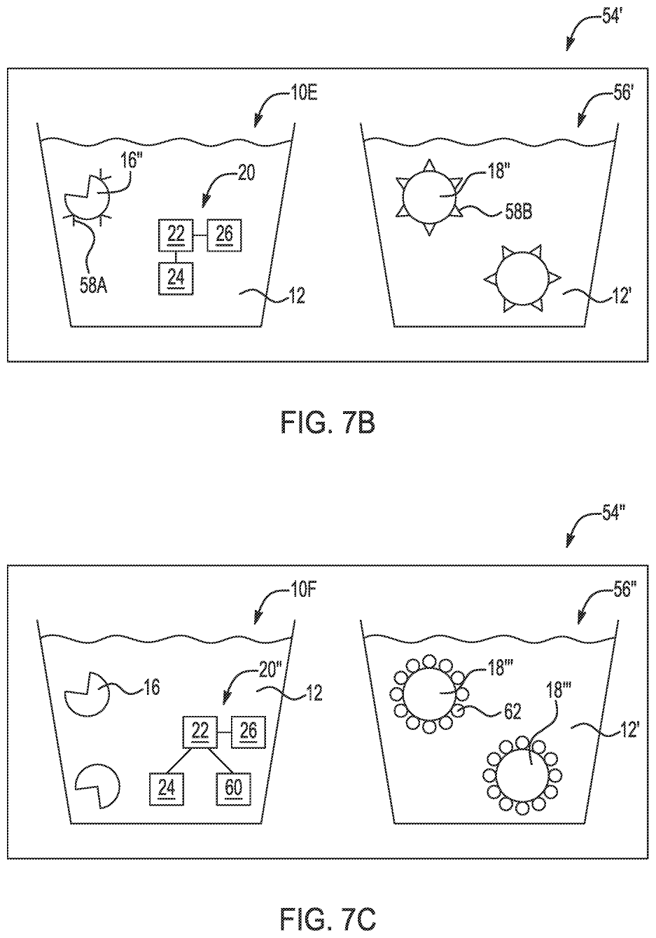

FIG. 7A, FIG. 7B, and FIG. 7C illustrate three examples of a kit 54, 54′, 54″ that include both an incorporation mix 10D, 10E, and 10F and an imaging mix 56, 56′, and 56″. Each kit 54, 54′, 54″ includes a different example of the incorporation mix 10D, 10E, and 10F, which includes the liquid carrier 12, an example of a polymerase 16 or 16″, and an example of the labeled nucleotide 20 or 20″. Each kit 54, 54′, 54″ also includes an example of the imaging mix 56, 56′, 56″, which includes a second liquid carrier 12′ and an example of a plasmonic nanostructure 18′, 18″, or 18′″ functionalized to associate itself within proximity of the labeled nucleotide 20 or 20″ after an incorporation event involving the labeled nucleotide 20 or 20″.

The liquid carrier 12 of the incorporation mixes 10D, 10E, and 10F and the second liquid carrier 12′ of the imaging mixes 56, 56′, 56″ may be water, and/or any of the buffers set forth herein. The liquid carrier 12′ may also include catalytic metal(s).

Each of these example kits 54, 54′, 54″ may be used in a sequencing method which generally involves introducing the incorporation mix 10D, 10E, and 10F to the flow cell including clusters of template strands 48, whereby at least one the polymerases 16 or 16″ incorporates an individual one of the labeled nucleotides 20 or 20″ into a nascent strand 52 along one the template strands 48; introducing the imaging mix 56 or 56′ or 56″ to the flow cell, whereby at least one the functionalized plasmonic nanostructures 18′, 18″, or 18′″ associates itself within proximity of the individual one of the labeled nucleotides 20 or 20″; and optically imaging the incorporation while the at least one the functionalized plasmonic nanostructures 18′, 18″, or 18′″ is associated with the individual one of the labeled nucleotides 20 or 20″.

Each of the kits 54, 54′, 54″ and its associated sequencing method will be further described, respectively, in reference FIG. 7A, FIG. 7B, and FIG. 7C.

In the example kit 54 shown in FIG. 7A, the incorporation mix 10D includes the polymerase 16 as it described in reference to FIG. 5A and the labeled nucleotide 20 as it described in reference to FIG. 1. The polymerase 16 may be any of the examples disclosed herein for use in incorporation, and may not be functionalized for being tethered to the template strand 48 (e.g., via the DNA binding domain) or to the flow cell surface 44 (e.g., via the surface tether 42). The polymerase 16 is used to incorporate the labeled nucleotide 20 during an incorporation event as described in reference to FIG. 6. It is to be understood that the incorporation mix 10D includes a plurality of the polymerases 16.

Also in the example kit 54 shown in FIG. 7A, the imaging mix 56 includes a plasmonic nanostructure 18′ that is functionalized with a second polymerase 16′. The plasmonic nanostructure 18′ functionalized with the second polymerase 16′ is similar to the complex 14, except that the second polymerase 16′ is not utilized for labeled nucleotide 20 incorporation, but rather is utilized for localizing the plasmonic nanostructure 18′ near the junction 46 between the template strand 48 and the most recently incorporated labeled nucleotide 20 after incorporation has taken place.

The second polymerase 16′ may be any example of the polymerase 16 set forth herein, or may be a polymerase that is inactive towards nucleotide incorporation. Any of the polymerases set forth herein may be exposed to active site mutations that can render the second polymerase 16′ unable to catalyze the incorporation reaction. The polymerases share a common mechanism using two active site aspartate residues, and mutating one or both of these residues renders the polymerize inactive. The second polymerase 16′ is also selected so that it can bind specifically to the 3′ end of the nascent strand 52.

Because the plasmonic nanostructure 18′ is linked to the second polymerase 16′ that binds specifically to the 3′ end of the nascent strand 52, the polymerase 16′ at least partially defines the space between the dye label 26 (of the incorporated nucleotide 20) and the plasmonic nanostructure 18′. As such, the size of the polymerase 16′ may be selected to create the signal enhancing proximity. In one example, the size of the polymerase 16′ ranges from about 3 nm to about 12 nm. In another example, the size of the polymerase 16′ ranges from about 4 nm to about 7 nm. In still another example, the size of the polymerase 16′ is about 5 nm. The size of the polymerase 16′ may be larger, e.g., up to about 100 nm when the distance associated with signal enhancing proximity is between 0.1 nm and 100 nm.

The second polymerase 16′ may be linked to the plasmonic nanostructure 18′ using the same techniques described herein to link the polymerase 16 to the plasmonic nanoparticle 18. In one example, the second polymerase 16′ may be linked to the plasmonic nanostructure 18′ using any of the chemical conjugation techniques set forth herein. In another example, the linkage similar to that shown in FIG. 2A is used where the oligonucleotide 28 is attached to the second polymerase 16′. In still another example, the linkage similar to that shown in FIG. 2B is used where the oligonucleotide 28 is attached to the second polymerase 16′ and the oligonucleotide tether 32 is wrapped around the plasmonic nanoparticle 18′.

The following description relates to an example method that involves the kit 54. This method also utilizes examples of the flow cell shown and described in reference to FIG. 8A, FIG. 8B and FIG. 8C. At the outset of this method, the template strands 48 have been formed as described in reference to FIG. 3.

The sequencing primer 50 may be introduced that hybridizes to a complementary portion of the sequence of the template strand 48. This sequencing primer 50 renders the template strand 48 ready for sequencing using the incorporation mix 10D and the imaging mix 56.

In this example method, the incorporation mix 10D is introduced into the flow cell, e.g., via an input port. When the incorporation mix 10D is introduced into the flow cell, the mix 10D enters a flow channel, and contacts the surface(s) 44 where the template strands 48 are present.

The incorporation mix 10D is allowed to incubate in the flow cell, and labeled nucleotides 20 are incorporated by respective polymerases 16. Similar to the example shown in FIG. 6, during incorporation, one of the labeled nucleotides 20 is incorporated, by a respective polymerase 16, into one nascent strand 52 that extends one sequencing primer 50 and that is complementary to one of the template strands 48. This incorporation is also performed in a template strand dependent fashion, and thus detection of the order and type of labeled nucleotides 20 added to the nascent strand 52 can be used to determine the sequence of the template strand 48. Incorporation occurs in at least some of the template strands 48 across the flow cell surface 44 during a single sequencing cycle.

The incorporated labeled nucleotides 20 include the reversible termination property due to the presence of the 3′ OH blocking group 24, which terminates further sequencing primer extension once the labeled nucleotide 20 has been added.

After a desired time for incubation and incorporation, the incorporation mix 10D, including non-incorporated labeled nucleotides 20, may be removed from the flow cell during a wash cycle. The incorporation mix 10D is removed prior to introducing the imaging mix 56. In this example, it is desirable for most, if not all, of the polymerases 16 from the incorporation mix 10D to be removed during the wash cycle. Multiple wash cycles may be performed in order to increase the percentage of polymerases 16 that are removed. The wash cycle(s) may involve a flow-through technique as described herein.

The imaging mix 56 is then introduced into the flow cell, e.g., via an input port. When the imaging mix 56 is introduced into the flow cell, the mix 56 enters a flow channel, and contacts the surface(s) 44 where the template strands 48, including the most recently incorporated labeled nucleotides 20, are present.

The imaging mix 56 is allowed to incubate in the flow cell, and at least some of the second polymerases 16′ bind specifically to the 3′ end of at least some of the nascent strands 52. Because each plasmonic nanostructure 18′ is functionalized with a second polymerase 16′, each plasmonic nanostructure 18′ is brought within signal enhancing proximity of a dye label 26 when the attached second polymerase 16′ binds specifically to the 3′ end of the nascent strand 52. Additionally, labeled nucleotides 20 are not introduced with the imaging mix 56, and thus additional labeled nucleotide 20 incorporation will not take place.

Without further incorporation taking place, the most recently incorporated labeled nucleotides 20 can be detected through an imaging event. During the imaging event, an illumination system (not shown) may provide an excitation light to the flow cell surface 44. The dye labels 26 of the incorporated labeled nucleotides 20 emit optical signals in response to the excitation light, and the respective plasmonic nanostructures 18′ secured by the second polymerases 16′ enhance these optical signals through plasmonic resonance.

After imaging is performed, a cleavage mix may then be introduced into the flow cell. In this example, the cleavage mix is capable of i) removing the 3′ OH blocking group 24 from the incorporated nucleotides 20, ii) cleaving the dye label 26 from the incorporated nucleotides 20, and iii) removing the second polymerase 16′ and the plasmonic nanoparticle 18′ attached thereto.

In this example, any of the de-blocking agents/components described herein for cleaving the 3′ OH blocking group 24 may be used. Also in this example, any of the cleaving agents/components described herein for cleaving the dye label 26 may be used.

Wash(es) may take place between the various fluid delivery steps. The sequencing cycle can then be repeated n times to extend the sequencing primer 50 by n nucleotides, thereby detecting a sequence of length n. In some examples, paired-end sequencing may be used, where the forward strands are sequenced and removed, and then reverse strands are constructed and sequenced.

In the example kit 54′ shown in FIG. 7B, the incorporation mix 10E includes the polymerase 16″, and the labeled nucleotide 20 as it described in reference to FIG. 1.

In this example, the polymerase 16″ is used for incorporation of labeled nucleotides 20 as well as for binding the plasmonic nanoparticle 18″, which is introduced in the imaging mix 56′. As such, the polymerase 16″ is any of the examples disclosed herein for use in nucleotide incorporation, and is also functionalized with one member 58A of a binding pair. As it is desirable for the polymerase 16″ to remain in place after incorporation, the polymerase 16″ may also be functionalized for being tethered to the template strand 48 (e.g., via the DNA binding domain) or to the flow cell surface 44 (e.g., via the surface tether 42). Any example of the surface tether 42 described herein may be used to anchor the polymerase 16″. It is to be understood that the incorporation mix 10E includes a plurality of the polymerases 16″.

The plasmonic nanostructure 18″ ultimately links to the polymerase 16″ that is used for incorporation, and thus the polymerase 16″ at least partially defines the space between the dye label 26 (of the incorporated nucleotide 20) and the plasmonic nanostructure 18″. As such, the size of the polymerase 16″ may be selected to create the signal enhancing proximity. In one example, the size of the polymerase 16″ ranges from about 3 nm to about 9 nm. In another example, the size of the polymerase 16″ ranges from about 4 nm to about 7 nm. In still another example, the size of the polymerase 16′ is about 5 nm.

Also in the example kit 54′ shown in FIG. 7B, the imaging mix 56′ includes a plasmonic nanostructure 18″ that is functionalized with another member 58B of the binding pair. The member 58B on the plasmonic nanoparticle 18″ is capable of binding to the member 58A on the polymerase 16″. As example binding pairs, the member 58B (on the plasmonic nanoparticle 18″) and the member 58A (on the polymerase 16″) respectively include a NiNTA (nickel-nitrilotriacetic acid) ligand and a histidine tag, or streptavidin or avidin and biotin, or a spytag and a spycatcher, or maleimide and cysteine, or azide and dibenzocyclooctyne (DBCO).

The following description relates to an example method that involves the kit 54′. This method also utilizes examples of the flow cell shown and described in detail in reference to FIG. 8A, FIG. 8B and FIG. 8C. At the outset of this method, the template strands 48 have been formed as described in reference to FIG. 3.

The sequencing primer 50 may be introduced that hybridizes to a complementary portion of the sequence of the template strand 48. This sequencing primer 50 renders the template strand 48 ready for sequencing using the incorporation mix 10E and the imaging mix 56′.

In this example method, the incorporation mix 10E is introduced into the flow cell, e.g., via an input port. When the incorporation mix 10E is introduced into the flow cell, the mix 10E enters a flow channel, and contacts the surface(s) 44 where the template strands 48 are present.

The incorporation mix 10E is allowed to incubate in the flow cell, and labeled nucleotides 20 are incorporated by respective polymerases 16″. Similar to the example shown in FIG. 6, during incorporation, one of the labeled nucleotides 20 is incorporated, by a respective polymerase 16″, into one nascent strand 52 that extends one sequencing primer 50 and that is complementary to one of the template strands 48. This incorporation is also performed in a template strand dependent fashion, and thus detection of the order and type of labeled nucleotides 20 added to the nascent strand 52 can be used to determine the sequence of the template strand 48. Incorporation occurs in at least some of the template strands 48 across the flow cell surface 44 during a single sequencing cycle.

The incorporated labeled nucleotides 20 include the reversible termination property due to the presence of the 3′ OH blocking group 24, which terminates further sequencing primer extension once the labeled nucleotide 20 has been added.

After a desired time for incubation and incorporation, the incorporation mix 10E, including non-incorporated labeled nucleotides 20, may be removed from the flow cell during a wash cycle. The incorporation mix 10E is removed prior to introducing the imaging mix 56′. At least some of the polymerases 16″ that perform incorporation remain in place at the junction 46 between the template strand 48 and the most recently incorporated labeled nucleotide 20 after the wash cycle. In one example, from about 20% to about 90% of the polymerases 16″ remain in the flow cell after the wash cycle. As mentioned herein, to increase the percentage of polymerases 16″ retained during and after the wash cycle, the polymerases 16″ may be tethered to the template strand 48 (e.g., via the DNA binding domain) or to the flow cell surface 44 (e.g., via the surface tether 42).

The imaging mix 56′ is then introduced into the flow cell, e.g., via an input port. When the imaging mix 56′ is introduced into the flow cell, the mix 56′ enters a flow channel, and contacts the surface(s) 44 where the template strands 48, including the most recently incorporated labeled nucleotides 20, are present.

The imaging mix 56′ is allowed to incubate in the flow cell, and at least some of the members 58B of the plasmonic nanostructure 18″ bind to the members 58A of the polymerases 16″ that remain at the junction 46 between the template strand 48 and the most recently incorporated labeled nucleotide 20. Therefore, the plasmonic nanostructure 18″ is brought within signal enhancing proximity of a dye label 26 when its member 58B binds specifically to the member 58A on the polymerase 16″.

Without further incorporation taking place, the most recently incorporated labeled nucleotides 20 can be detected through an imaging event. During the imaging event, an illumination system (not shown) may provide an excitation light to the flow cell surface 44. The dye labels 26 of the incorporated labeled nucleotides 20 emit optical signals in response to the excitation light, and the respective plasmonic nanostructures 18″ secured by the polymerases 16″ enhance these optical signals through plasmonic resonance.

After imaging is performed, a cleavage mix may then be introduced into the flow cell. In this example, the cleavage mix is capable of i) removing the 3′ OH blocking group 24 from the incorporated nucleotides 20, ii) cleaving the dye label 26 from the incorporated nucleotides 20, and iii) removing the polymerases 16″ and the plasmonic nanoparticle 18″ attached thereto.

In this example, any of the de-blocking agents/components described herein for cleaving the 3′ OH blocking group 24 may be used. Also in this example, any of the cleaving agents/components described herein for cleaving the dye label 26 may be used. When the polymerases 16″ are linked to the template strand 48 (e.g., via the DNA binding domain) or to the flow cell surface 44 (e.g., via the surface tether 42), an additional polymerase-removing agent may be added to the cleavage mix as described herein. The polymerase-removing agent will depend upon the linkage formed between the polymerase 16″ and the template strand 48 or between the polymerase 16″ and the flow cell surface 44. In one example, a biotin-streptavidin binding pair links the surface tether 42 (and the polymerase 16″) to the flow cell surface 44. In this example, the polymerase-removing agent may be sodium dodecyl sulfate (SDS), a combination of urea and biotin, or a combination of from about 10% by volume to about 50% by volume of a formamide reagent including formamide and an optional buffer and a balance of a salt buffer including sodium chloride, sodium citrate, and a biocompatible surfactant.

Wash(es) may take place between the various fluid delivery steps. The sequencing cycle can then be repeated n times to extend the sequencing primer 50 by n nucleotides, thereby detecting a sequence of length n. In some examples, paired-end sequencing may be used, where the forward strands are sequenced and removed, and then reverse strands are constructed and sequenced.

In the example kit 54″ shown in FIG. 7C, the incorporation mix 10F includes the polymerase 16 as described in FIG. 5A, and another example of the labeled nucleotide 20″.

In this example of the incorporation mix 10F, the polymerase 16 may be any of the examples disclosed herein for use in incorporation, and may not be functionalized for being tethered to the template strand 48 (e.g., via the DNA binding domain) or to the flow cell surface 44 (e.g., via the surface tether 42). The polymerase 16 is used to incorporate the labeled nucleotide 20″ during an incorporation event as described in reference to FIG. 6. It is to be understood that the incorporation mix 10F includes a plurality of the polymerases 16.

The labeled nucleotide 20″ includes the nucleotide 22, the 3′ OH blocking group 24 attached to a sugar of the nucleotide 22, the dye label 26 attached to a base of the nucleotide 22. The labeled nucleotide 20″ is also biotinylated, and thus includes a biotin label 60. The biotin label 60 may be added to the base of the labeled nucleotide 20″ through a suitable linking molecule, such as a spacer group of formula —((CH₂)₂O)ₙ— wherein n is an integer between 2 and 50, a PEG linker, or any of the linking molecule 38A, 38B, 38C shown in FIG. 4. The biotin label 60 may alternatively be incorporated just past the dye label 26 (i.e., attached to the dye label 26).

The plasmonic nanostructure 18′″ ultimately links to the biotin label 60 of the labeled nucleotide 20″, and thus the length of the linking molecule attaching the biotin label 60 may be selected so that the bound plasmonic nanostructure 18′″ is within signal enhancing proximity of the dye label 26 of the labeled nucleotide 20″. In one example, the biotin label's linking molecule has a length that is within from about 3 nm to about 12 nm of a length of the dye label's linking molecule 38. In another example, the biotin label's linking molecule has a first length, the dye label's linking molecule 38 has a second length, and together the first and second lengths range from about 3 nm to about 12 nm.

Also in the example kit 54″ shown in FIG. 7C, the imaging mix 56″ includes a plasmonic nanostructure 18′″ that is functionalized with streptavidin 62. The streptavidin 62 on the plasmonic nanostructure 18′″ and the biotin label 60 on the labeled nucleotide 20″ form a binding pair.

The following description relates to an example method that involves the kit 54″. This method also utilizes examples of the flow cell shown and described in detail in reference to FIG. 8A, FIG. 8B and FIG. 8C. At the outset of this method, the template strands 48 have been formed as described in reference to FIG. 3.

The sequencing primer 50 may be introduced that hybridizes to a complementary portion of the sequence of the template strand 48. This sequencing primer 50 renders the template strand 48 ready for sequencing using the incorporation mix 10F and the imaging mix 56″.

In this example method, the incorporation mix 10F is introduced into the flow cell, e.g., via an input port. When the incorporation mix 10F is introduced into the flow cell, the mix 10F enters a flow channel, and contacts the surface(s) 44 where the template strands 48 (with the sequencing primer 50 hybridized thereto) are present.

The incorporation mix 10F is allowed to incubate in the flow cell, and labeled nucleotides 20″ are incorporated by respective polymerases 16. Similar to the example shown in FIG. 6, during incorporation, one of the labeled nucleotides 20″ is incorporated, by a respective polymerase 16, into one nascent strand 52 that extends one sequencing primer 50 and that is complementary to one of the template strands 48. This incorporation is also performed in a template strand dependent fashion, and thus detection of the order and type of labeled nucleotides 20″ added to the nascent strand 52 can be used to determine the sequence of the template strand 48. Incorporation occurs in at least some of the template strands 48 across the flow cell surface 44 during a single sequencing cycle.

The incorporated labeled nucleotides 20″ include the reversible termination property due to the presence of the 3′ OH blocking group 24, which terminates further sequencing primer extension once the labeled nucleotide 20″ has been added.

After a desired time for incubation and incorporation, the incorporation mix 10F, including non-incorporated labeled nucleotides 20″, may be removed from the flow cell during a wash cycle. The incorporation mix 10F is removed prior to introducing the imaging mix 56″. In this example, it is desirable for most, if not all, of the polymerases 16 from the incorporation mix 10F to be removed during the wash cycle. Multiple wash cycles may be performed in order to increase the percentage of polymerases 16 that are removed. The wash cycle(s) may involve a flow-through technique as described herein.

The imaging mix 56″ is then introduced into the flow cell, e.g., via an input port. When the imaging mix 56″ is introduced into the flow cell, the mix 56″ enters a flow channel, and contacts the flow cell surface(s) 44 where the template strands 48, including the most recently incorporated labeled nucleotides 20″, are present.

The imaging mix 56″ is allowed to incubate in the flow cell, and at least some of the plasmonic nanoparticles 18′″ specifically bind, through the streptavidin 62 at their surface, to the biotin labels 60 of at least some of the incorporated nucleotides 20″. Because the biotin label 60 is part of the labeled nucleotide 20″ to which the plasmonic nanoparticle 18′″ binds, the plasmonic nanostructure 18′ is brought within signal enhancing proximity of the dye label 26 of that labeled nucleotides 20″.

Without further incorporation taking place, the most recently incorporated labeled nucleotides 20″ can be detected through an imaging event. During the imaging event, an illumination system (not shown) may provide an excitation light to the flow cell surface 44. The dye labels 26 of the incorporated labeled nucleotides 20″ emit optical signals in response to the excitation light, and the respective plasmonic nanostructures 18′″, which are secured by the interaction between the streptavidin 62 and the biotin label 60, enhance these optical signals through plasmonic resonance.

After imaging is performed, a cleavage mix may then be introduced into the flow cell. In this example, the cleavage mix is capable of i) removing the 3′ OH blocking group 24 from the incorporated nucleotides 20″, ii) cleaving the dye label 26 from the incorporated nucleotides 20″, and iii) cleaving the biotin label 60 from the incorporated nucleotides 20″.

In this example, any of the de-blocking agents/components described herein for cleaving the 3′ OH blocking group 24 may be used. Also in this example, any of the cleaving agents/components described herein for cleaving the dye label 26 may be used. When the biotin label linking molecule is the same as the dye label linking molecule 38, the cleavage mix may include a single cleaving agent/component that can cleave both the biotin label 60 and the dye label 26. When the biotin label linking molecule is different than the dye label linking molecule 38, the cleavage mix may include different cleaving agents/components that can cleave the respective linking molecules.

Wash(es) may take place between the various fluid delivery steps. The sequencing cycle can then be repeated n times to extend the sequencing primer 50 by n nucleotides, thereby detecting a sequence of length n. In some examples, paired-end sequencing may be used, where the forward strands are sequenced and removed, and then reverse strands are constructed and sequenced.

Flow Cells

Examples of the flow cells that may be used in the examples disclosed herein will now be described in reference to FIG. 8A, FIG. 8B, and FIG. 8C.

A top view of an example of the flow cell 64 is shown in FIG. 8A. As will be discussed in reference to FIG. 8B and FIG. 8C, some examples of the flow cell 64 include two opposed flow cell surfaces 44 where sequencing can take place. An example of non-patterned sequencing surfaces 44A, 44A′ are shown in FIG. 8B, and an example of patterned sequencing surfaces 44B, 44B′ are shown in FIG. 8C. Each sequencing surface 44A, 44A′ or 44B, 44B′ is supported by a substrate (generally shown as 66 in FIG. 8A); and a flow channel (generally shown as 68 in FIG. 8A) is defined between the sequencing surfaces 44A, 44A′ or 44B, 44B′. In other examples, the flow cell 64 includes one sequencing surface 44A or 44B supported by a substrate 66 and a lid attached to the substrate 66. In these examples, the flow channel 68 is defined between the sequencing surface 44A or 44B and the lid.

In any of the examples, the substrate 66 may be a single layer/material. Examples of the single layer substrate are shown at reference numeral 66A and 66A′ in FIG. 8B. Examples of suitable single layer substrates 66A, 66A′ include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyim ides, etc.), nylon (polyam ides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

The substrate 66 may also be a multi-layered structure. Examples of the multi-layered substrate are shown at reference numeral 66B and 66B′ in FIG. 8C. Some examples of the multi-layered structure 66B, 66B′ include glass or silicon, with a coating layer of tantalum pentoxide or another oxide that is transparent to the light used in optical imaging. With specific reference to FIG. 8C, other examples of the multi-layered structure 66B, 66B′ include an underlying support 70, 70′ having a patterned resin 72, 72′ thereon. Still other examples of the multi-layered substrate 66B, 66B′ may include a silicon-on-insulator (SOI) substrate.

In an example, the substrate 66 (whether single or multi-layered) may be round and have a diameter ranging from about 2 mm to about 300 mm, or may be a rectangular sheet or panel having its largest dimension up to about 10 feet (˜3 meters). In an example, the substrate 26 is a wafer having a diameter ranging from about 200 mm to about 300 mm. In another example, the substrate 66 is a die having a width ranging from about 0.1 mm to about 10 mm. While example dimensions have been provided, it is to be understood that a substrate 66 with any suitable dimensions may be used. For another example, a panel may be used that is a rectangular support, which has a greater surface area than a 300 mm round wafer.

In the example shown in FIG. 8A, the flow cell 64 includes flow channels 68. While several flow channels 68 are shown, it is to be understood that any number of channels 68 may be included in the flow cell 64 (e.g., a single channel 68, four channels 68, etc.). In some of the examples disclosed herein, each flow channel 68 is an area defined between two sequencing surfaces (e.g., 44A and 44A′ or 44B and 44B′) and by two attached substrates (e.g., 66A and 66A′ or 66B and 66B′). In other of the examples disclosed herein, each flow channel 68 is an area defined between one sequencing surface (e.g., 44A or 44B) and a lid. The incorporation mixes 10A-10F, the imaging mixes 56, 56′, 56″, and other fluids described herein can be introduced into and removed from the flow channel(s) 68. Each flow channel 68 may be isolated from each other flow channel 68 in a flow cell 64 so that fluid introduced into any particular flow channel 68 does not flow into any adjacent flow channel 68.

A portion of the flow channel 68 may be defined in the substrate 66 using any suitable technique that depends, in part, upon the material(s) of the substrate 66. In one example, a portion of the flow channel 68 is etched into a glass substrate, such as substrate 66A, 66A′. In another example, a portion of the flow channel 68 may be patterned into a resin 72, 72′ of a multi-layered substrate 66B, 66B′ using photolithography, nanoimprint lithography, etc. A separate material (e.g., material 74 in FIG. 8B and FIG. 8C) may be applied to the substrate 66 so that the separate material defines at least a portion of the walls of the flow channel 68.

In an example, the flow channel 68 has a substantially rectangular configuration with rounded ends. The length and width of the flow channel 68 may be smaller, respectively, than the length and width of the substrate 66 so that a portion of the substrate surface surrounding the flow channel 68 is available for attachment to another substrate 66 or a lid. In some instances, the width of each flow channel 68 can be at least about 1 mm, at least about 2.5 mm, at least about 5 mm, at least about 7 mm, at least about 10 mm, or more. In some instances, the length of each flow channel 68 can be at least about 10 mm, at least about 25 mm, at least about 50 mm, at least about 100 mm, or more. The width and/or length of each flow channel 68 can be greater than, less than or between the values specified above. In another example, the flow channel 68 is square (e.g., 10 mm×10 mm).

The depth of each flow channel 68 can be as small as a few monolayers thick, for example, when microcontact, aerosol, or inkjet printing is used to deposit a separate material 74 that defines the flow channel walls. In other examples, the depth of each flow channel 68 can be about 1 μm, about 10 μm, about 50 μm, about 100 μm, or more. In an example, the depth may range from about 10 μm to about 100 μm. In another example, the depth is about 5 μm or less. It is to be understood that the depth of each flow channel 68 can also be greater than, less than or between the values specified above. The depth of the flow channel 68 may also vary along the length and width of the flow cell 64, e.g., when a patterned sequencing surface 44B, 44B′ is used.

FIG. 8B illustrates a cross-sectional view of the flow cell 64 including non-patterned opposed sequencing surfaces 44A, 44A′. In an example, each of these surfaces 44A, 44A′ may be prepared on the substrate 66A, 66A′, and then the substrates 44A, 44A′ may be attached to one another to form an example of the flow cell 64. Any suitable bonding material 74, such as an adhesive, a radiation-absorbing material that aids in bonding, etc., may be used to bond the substrates 66A, 66A′ together.

In the example shown in FIG. 8B, a portion of the flow channel 68 is defined in each of the single layer substrates 66A, 66A′. For example, each substrate 66A, 66A′ may have a concave region 76A, 76A′ defined therein where the components of the sequencing surface 44A, 44A′ may be introduced. It is to be understood that any space within the concave region 76A, 76A′ not occupied by the components of the sequencing surface 44A, 44A′ may be considered to be part of the flow channel 68.

The sequencing surfaces 44A, 44A′ include a polymeric hydrogel 78A, 78A′ and amplification primers 80A, 82A or 80A′, 82A′ attached to the polymeric hydrogel 78A, 78A″. While not shown, some examples of the sequencing surfaces 44A, 44A′ may also include a binding agent (e.g., streptavidin, avidin, biotin, etc.) that can attach to the surface tether 42. This binding agent may be present in the concave regions 76A, 76A′.

An example of the polymeric hydrogel 78A, 78A′ includes an acrylamide copolymer, such as poly(N-(5-azidoacetamidylpentyl)acrylamide-co-acrylamide, PAZAM. PAZAM and some other forms of the acrylamide copolymer are represented by the following structure (I):

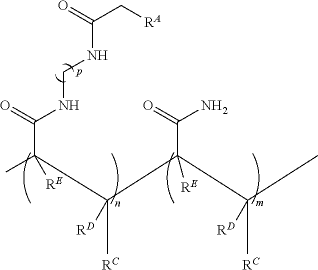

wherein:

R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;

R^{B} is H or optionally substituted alkyl;

R^{C}, R^{D}, and R^{E} are each independently selected from the group consisting of H and optionally substituted alkyl;

each of the —(CH₂)ₚ— can be optionally substituted;

p is an integer in the range of 1 to 50;

n is an integer in the range of 1 to 50,000; and

m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In other examples, the polymeric hydrogel 78A, 78A′ may be a variation of the structure (I). In one example, the acrylamide unit may be replaced with N,N-dimethylacrylamide

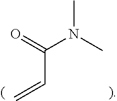

In this example, the acrylamide unit in structure (I) may be replaced with

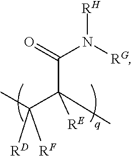

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

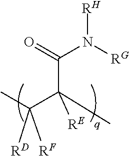

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 78A, 78A′ the recurring “n” feature in structure (I) may be replaced with a monomer including a heterocyclic azido group having structure (II):

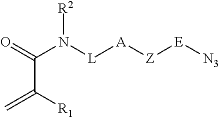

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 ring members present as a single cyclic structure or a fused structure. Some specific examples of Z include pyrrolidinyl, pyridinyl, or pyrimidinyl.

As still another example, the polymeric hydrogel 78A, 78A′ may include a recurring unit of each of structure (III) and (IV):

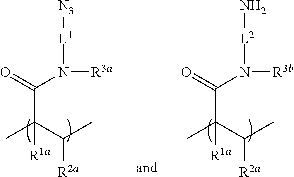

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

It is to be understood that other monomers may be used to form the polymeric hydrogel 78A, 78A′, as long as they are functionalized to graft amplification primers 80A, 82A or 80A′, 82A′ thereto. Other examples of suitable polymer layers include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels 78A, 78A′ include mixed copolymers of acrylamides and acrylates. A variety of polymer architectures containing acrylic monomers (e.g., acrylamides, acrylates etc.) may be utilized in the examples disclosed herein, such as branched polymers, including star polymers, star-shaped or star-block polymers, dendrimers, and the like. For example, the monomers (e.g., acrylamide, etc.) may be incorporated, either randomly or in block, into the branches (arms) of a star-shaped polymer.

To introduce the polymeric hydrogel 78A, 78A′ into the concave regions 76A, 76A′, a mixture of the polymeric hydrogel 78A, 78A′ may be generated and then applied to the respective substrates 66A, 66A′ (having the concave regions 76A, 76A′ defined therein). In one example, the polymeric hydrogel 78A, 78A′ may be present in a mixture (e.g., with water or with ethanol and water). The mixture may then be applied to the respective substrate surfaces (including in the concave regions 76A, 76A′) using spin coating, or dipping or dip coating, or flow of the material under positive or negative pressure, or another suitable technique. These types of techniques blanketly deposit the polymeric hydrogel 78A, 78A′ on the substrate respective substrates 66A, 66A′ (e.g., in the concave regions 76A, 76A′ and on interstitial regions 84A, 84A′ adjacent thereto). Other selective deposition techniques (e.g. involving a mask, controlled printing techniques, etc.) may be used to specifically deposit the polymeric hydrogel 78A, 78A′ in the concave regions 76A, 76A′ and not on the interstitial regions 84A, 84A′.

In some examples, the substrate surface (including the concave regions 76A, 76A′) may be activated, and then the mixture (including the polymeric hydrogel 78A, 78A′ may be applied thereto. In one example, a silane or silane derivative (e.g., norbornene silane) may be deposited on the substrate surface using vapor deposition, spin coating, or other deposition methods. In another example, the substrate surface may be exposed to plasma ashing to generate surface-activating agent(s) (e.g., —OH groups) that can adhere to the polymeric hydrogel 78A, 78A′.

Depending upon the chemistry of the polymeric hydrogel 78A, 78A′, the applied mixture may be exposed to a curing process. In an example, curing may take place at a temperature ranging from room temperature (e.g., about 25° C.) to about 95° C. for a time ranging from about 1 millisecond to about several days.

Polishing may then be performed in order to remove the polymeric hydrogel 78A, 78A′ from the interstitial regions 84A, 84A′ at the perimeter of the concave regions 76A, 76A′, while leaving the polymeric hydrogel 78A, 78A′ on the surface in the concave regions 76A, 76A′ at least substantially intact.

The sequencing surfaces 44A, 44A′ also include amplification primers 80A, 82A or 80A′, 82A′ attached to the polymeric hydrogel 78A, 78A′.

A grafting process may be performed to graft the amplification primers 80A, 82A or 80A′, 82A′ to the polymeric hydrogel 78A, 78A′ in the concave regions 76A, 76A′. In an example, the amplification primers 80A, 82A or 80A′, 82A′ can be immobilized to the polymeric hydrogel 78A, 78A′ by single point covalent attachment at or near the 5′ end of the primers 80A, 82A or 80A′, 82A′. This attachment leaves i) an adapter-specific portion of the primers 80A, 82A or 80A′, 82A′ free to anneal to its cognate sequencing-ready nucleic acid fragment and ii) the 3′ hydroxyl group free for primer extension. Any suitable covalent attachment may be used for this purpose. Examples of terminated primers that may be used include alkyne terminated primers (e.g., which may attach to an azide surface moiety of the polymeric hydrogel 78A, 78A′), or azide terminated primers (e.g., which may attach to an alkyne surface moiety of the polymeric hydrogel 78A, 78A′).

Specific examples of suitable primers 80A, 82A or 80A′, 82A′ include P5 and P7 primers used on the surface of commercial flow cells sold by IIlumina Inc. for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms. Both P5 and P7 primers may be grafted to each of the polymeric hydrogels 78A, 78A′.

In an example, grafting may involve flow through deposition (e.g., using a temporarily bound lid), dunk coating, spray coating, puddle dispensing, or by another suitable method that will attach the primer(s) 80A, 82A or 80A′, 82A′ to the polymeric hydrogel 78A, 78A′. Each of these example techniques may utilize a primer solution or mixture, which may include the primer(s) 80A, 82A or 80A′, 82A′, water, a buffer, and a catalyst. With any of the grafting methods, the primers 42, 42′ react with reactive groups of the polymeric hydrogel 78A, 78A′ in the concave region 76A, 76A′ and have no affinity for the surrounding substrate 66A, 66A′. As such, the primers 80A, 82A or 80A′, 82A′ selectively graft to the polymeric hydrogel 78A, 78A′.

FIG. 8C illustrates a cross-sectional view of the flow cell 64 including patterned opposed sequencing surfaces 44B, 44B′. In an example, each of these surfaces 44B, 44B′ may be prepared on the substrate 66B, 66B′, and then the substrates 66B, 66B′ may be attached to one another (e.g., via material 74) to form an example of the flow cell 64.

In the example shown in FIG. 8C, the flow cell 64 includes the multi-layer substrate 66B, 66B′, each of which includes the support 70, 70′ and the patterned material 72, 72′ positioned on the support 70, 70′. The patterned material 72, 72′ defines depressions 88, 88′ separated by interstitial regions 84B, 84B′.

In the example shown in FIG. 8C, the patterned material 72, 72′ is respectively positioned on the support 70, 70′. It is to be understood that any material that can be selectively deposited, or deposited and patterned to form the depressions 88, 88′ and the interstitial regions 84B, 84B′ may be used for the patterned material 72, 72′.

As one example, an inorganic oxide may be selectively applied to the support 70, 70′ via vapor deposition, aerosol printing, or inkjet printing. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

As another example, a resin may be applied to the support 70, 70′ and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (commercially available under the tradename “POSS” from Hybrid Plastics) refers to a chemical composition that is a hybrid intermediate (e.g., RSiO_{1.5}) between that of silica (SiO₂) and silicone (R₂SiO). An example of polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units. The average cage content can be adjusted during the synthesis, and/or controlled by purification methods, and a distribution of cage sizes of the monomeric unit(s) may be used in the examples disclosed herein.

As shown in FIG. 8C, the patterned material 72, 72′ includes the depressions 88, 88′ respectively defined therein, and interstitial regions 84B, 84B′ separating adjacent depressions 88, 88′. Many different layouts of the depressions 88, 88′ may be envisaged, including regular, repeating, and non-regular patterns. In an example, the depressions 88, 88′ are disposed in a hexagonal grid for close packing and improved density. Other layouts may include, for example, rectilinear (rectangular) layouts, triangular layouts, and so forth. In some examples, the layout or pattern can be an x-y format of depressions 88, 88′ that are in rows and columns. In some other examples, the layout or pattern can be a repeating arrangement of depressions 88, 88′ and/or interstitial regions 84B, 84B′. In still other examples, the layout or pattern can be a random arrangement of depressions 88, 88′ and/or interstitial regions 84B, 84B′. The pattern may include stripes, swirls, lines, triangles, rectangles, circles, arcs, checks, diagonals, arrows, and/or squares.

The layout or pattern of the depressions 88, 88′ may be characterized with respect to the density of the depressions 88, 88′ (e.g., number of depressions 88, 88′) in a defined area. For example, the depressions 88, 88′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of depressions 88, 88′ in the patterned material 72, 72′ can be between one of the lower values and one of the upper values selected from the ranges above. As examples, a high density array may be characterized as having depressions 88, 88′ separated by less than about 100 nm, a medium density array may be characterized as having depressions 88, 88′ separated by about 400 nm to about 1 μm, and a low density array may be characterized as having depressions 88, 88′ separated by greater than about 1 μm. While example densities have been provided, it is to be understood that any suitable densities may be used. The density of the depressions 88, 88′ may depend, in part, on the depth of the depressions 88, 88′. In some instances, it may be desirable for the spacing between depressions 88, 88′ to be even greater than the examples listed herein.

The layout or pattern of the depressions 88, 88′ may also or alternatively be characterized in terms of the average pitch, or the spacing from the center of the depression 88, 88′ to the center of an adjacent depression 88, 88′ (center-to-center spacing) or from the left edge of one depression 88, 88′ to the right edge of an adjacent depression 88, 88′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of depressions 88, 88′ can be between one of the lower values and one of the upper values selected from the ranges above. In an example, the depressions 88, 88′ have a pitch (center-to-center spacing) of about 1.5 μm. While example average pitch values have been provided, it is to be understood that other average pitch values may be used.

The size of each depression 88, 88′ may be characterized by its volume, opening area, depth, and/or diameter.

Each depression 88, 88′ can have any volume that is capable of confining at least some fluid that is introduced into the flow cell 64. The minimum or maximum volume can be selected, for example, to accommodate the throughput (e.g., multiplexity), resolution, nucleotides, or analyte reactivity expected for downstream uses of the flow cell 64. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area occupied by each depression opening can be selected based upon similar criteria as those set forth above for the volume. For example, the area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 88, 88′ can be large enough to house some of the polymeric hydrogel 78B, 78B′. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 88, 88′ can be greater than, less than or between the values specified above.

In some instances, the diameter or length and width of each depression 88, 88′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or length and width is about 0.4 μm. The diameter or length and width of each depression 88, 88′ can be greater than, less than or between the values specified above.

In this example, at least some of components of the sequencing surface 44B, 44B′ may be introduced into the depressions 88, 88′. It is to be understood that any space within the depressions 88, 88′ not occupied by the components of the sequencing surface 44B, 44B′ may be considered to be part of the flow channel 68.

In the example shown in FIG. 8C, the polymeric hydrogel 78B, 78B′ is positioned within each of the depressions 88, 88′. The polymeric hydrogel 78B, 78B′ may be any of the examples set forth herein for the polymeric hydrogel 78A, 78A′, and may applied as described in reference to FIG. 8B (e.g., deposition followed by polishing), so that the polymeric hydrogel 78B, 78B′ is present in the depressions 88, 88′ and not present on the surrounding interstitial regions 84B, 84B′.

In the example shown in FIG. 8C, the primers 80B, 82B or 80B′, 82B′ may be any of the examples set forth herein for the primers 80A, 82A or 80A′, 82A′, and may be grafted to the polymeric hydrogel 78B, 78B′ within each of the depressions 88, 88′. The primers 80B, 82B or 80B′, 82B′ may be applied as described in reference to FIG. 8B, and thus will graft to the polymeric hydrogel 78B, 78B′ and not to the surrounding interstitial regions 84B, 84B′.

While not shown, some examples of the sequencing surfaces 44B, 44B′ may also include a binding agent (e.g., streptavidin, avidin, biotin, etc.) that can attach the surface tether 42. This binding agent may be present in the depressions 88, 88′.

As shown in FIG. 8B and FIG. 8C, the substrates 66A and 66A′ or 66B and 66B′ are attached to one another so that the sequencing surfaces 44A and 44A′ or 44B and 44B′ face each other with the flow channel 68 defined therebetween.

The substrates 66A and 66A′ or 66B and 66B′ may be bonded to each other at some or all of the interstitial regions 84A and 84A′ or 84B and 84B′. The bond that is formed between the substrates 66A and 66A′ or 66B and 66B′ may be a chemical bond, or a mechanical bond (e.g., using a fastener, etc.).

Any suitable technique, such as laser bonding, diffusion bonding, anodic bonding, eutectic bonding, plasma activation bonding, glass frit bonding, or other methods known in the art may be used to bond the substrates 66A and 66A′ or 66B and 66B′ together. In an example, a spacer layer (e.g., material 74) may be used to bond the substrates 66A and 66A′ or 66B and 66B′. The spacer layer may be any material 74 that will seal at least some portion of the substrates 66A and 66A′ or 66B and 66B′ together. In some examples, the spacer layer can be a radiation-absorbing material that aids in bonding.

While not shown, it is to be understood that a lid may be bonded to one of the substrate 66A or 66B so that the flow cell has one sequencing surface.

### ADDITIONAL NOTES

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range from about 2 mm to about 300 mm, should be interpreted to include not only the explicitly recited limits of from about 2 mm to about 300 mm, but also to include individual values, such as about 40 mm, about 250.5 mm, etc., and sub-ranges, such as from about 25 mm to about 175 mm, etc.

Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. An incorporation mix, comprising:
a liquid carrier;
a complex, including:
a polymerase; and
a plasmonic nanostructure linked to the polymerase; and
a labeled nucleotide, including:
a nucleotide;
a 3′ OH blocking group attached to a sugar of the nucleotide; and
a dye label attached to a base of the nucleotide.

2. The incorporation mix as defined in claim 1, wherein the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

3. The incorporation mix as defined in claim 1, wherein the plasmonic nanostructure is chemically conjugated to an amine or a cysteine of the polymerase.

4. The incorporation mix as defined in claim 1, wherein:
an oligonucleotide is attached to the polymerase; and
the oligonucleotide is hybridized to a complementary oligonucleotide tether that is attached to the plasmonic nanostructure.

5. The incorporation mix as defined in claim 1, wherein:
an oligonucleotide is attached to the polymerase; and
the oligonucleotide is hybridized to a complementary portion of an oligonucleotide tether that also includes an additional portion that is wrapped around the plasmonic nanostructure.

6. The incorporation mix as defined in claim 1, wherein:
the plasmonic nanostructure is functionalized with a first member of a binding pair; and
the polymerase includes or is functionalized with a second member of the binding pair.

7. The incorporation mix as defined in claim 6, wherein the first member and the second member include a NiNTA ligand and a histidine tag, or streptavidin and biotin, or a spytag and a spycatcher, or maleimide and cysteine, or azide and dibenzocyclooctyne.

8. A method, comprising:
introducing an incorporation mix to a flow cell including clusters of template strands, the incorporation mix including:
a liquid carrier;
a plurality of complexes, each complex including:
a polymerase; and
a plasmonic nanostructure linked to the polymerase; and
a plurality of labeled nucleotides, each labeled nucleotide including:
a nucleotide;
a 3′ OH blocking group attached to a sugar of the nucleotide; and
a dye label attached to a base of the nucleotide;
whereby at least one the polymerases i) incorporates an individual one of the labeled nucleotides into a nascent strand along one the template strands, and ii) maintains its linked plasmonic nanostructure within proximity of the individual one of the labeled nucleotides; and
optically imaging the incorporation while the plasmonic nanostructure is maintained.

9. A kit, comprising:
an incorporation mix, including:
a liquid carrier;
a polymerase; and
a labeled nucleotide including:
a nucleotide;
a 3′ OH blocking group attached to a sugar of the nucleotide; and
a dye label attached to a base of the nucleotide; and
an imaging mix, including:
a second liquid carrier; and
a plasmonic nanostructure functionalized to associate itself within proximity of the labeled nucleotide after an incorporation event involving the labeled nucleotide.

10. The kit as defined in claim 9, wherein the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

11. The kit as defined in claim 9, wherein the plasmonic nanostructure is functionalized with a second polymerase.

12. The kit as defined in claim 11, wherein the plasmonic nanostructure is chemically conjugated to an amine or a cysteine of the second polymerase.

13. The kit as defined in claim 11, wherein:
an oligonucleotide is attached to the second polymerase; and
the oligonucleotide is hybridized to a complementary oligonucleotide tether that is attached to the plasmonic nanostructure.

14. The kit as defined in claim 11, wherein:
an oligonucleotide is attached to the second polymerase; and
the oligonucleotide is hybridized to a complementary oligonucleotide tether that includes a portion that is wrapped around the plasmonic nanostructure.

15. The kit as defined in claim 9, wherein:
the plasmonic nanostructure is functionalized with a first member of a binding pair; and
the polymerase includes or is functionalized with a second member of the binding pair.

16. The kit as defined in claim 15, wherein the first member and the second member include a NiNTA ligand and a histidine tag, or streptavidin and biotin, or a spytag and a spycatcher, or maleimide and cysteine, or azide and dibenzocyclooctyne.

17. The kit as defined in claim 15, wherein the polymerase further comprises a DNA binding domain attached to a surface thereof.

18. The kit as defined in claim 15, wherein the polymerase further comprises:
a surface tether attached to a surface thereof; and
a flow cell surface binding agent attached to the surface tether.

19. The kit as defined in claim 9, wherein:
the plasmonic nanostructure is functionalized with streptavidin; and
the labeled nucleotide is biotinylated.

20. A method, comprising:
introducing an incorporation mix to a flow cell including clusters of template strands, the incorporation mix including:
a liquid carrier;
a plurality of polymerases; and
a plurality of labeled nucleotides, each labeled nucleotide including:
a nucleotide;
a 3′ OH blocking group attached to a sugar of the nucleotide; and
a dye label attached to a base of the nucleotide;
whereby at least one the polymerases incorporates an individual one of the labeled nucleotides into a nascent strand along one the template strands;
introducing an imaging mix into the flow cell, the imaging mix including:
a second liquid carrier; and
a plurality of functionalized plasmonic nanostructures;
whereby at least one of the functionalized plasmonic nanostructures associates itself within proximity of the individual one of the labeled nucleotides; and
optically imaging the incorporation while the at least one of the functionalized plasmonic nanostructures is associated with the individual one of the labeled nucleotides.

21. The method as defined in claim 20, wherein:
each of the functionalized plasmonic nanostructures is functionalized with a second polymerase; and
the method further comprises removing the incorporation mix prior to introducing the imaging mix.

22. The method as defined in claim 20, wherein:
each of the functionalized plasmonic nanostructures is functionalized with a first member of a binding pair;
each of the polymerases includes a second member of the binding pair; and
the method further comprises removing the incorporation mix prior to introducing the imaging mix.

23. The method as defined in claim 20, wherein:
each of the functionalized plasmonic nanostructures is functionalized with streptavidin;
each of the labeled nucleotides is biotinylated; and
the method further comprises removing the incorporation mix prior to introducing the imaging mix.

24. A labeled nucleotide, comprising:
a nucleotide;
a 3′ OH blocking group attached to a sugar of the nucleotide;
a dye label attached to a base of the nucleotide; and
a plasmonic nanostructure attached to the base of the nucleotide or to the dye label.

25. The labeled nucleotide as defined in claim 24, wherein the plasmonic nanostructure is selected from the group consisting of a gold nanostructure, a silver nanostructure, a tin nanostructure, a rhodium nanostructure, a ruthenium nanostructure, a palladium nanostructure, an osmium nanostructure, an iridium nanostructure, a platinum nanostructure, a chromium nanostructure, a copper nanostructure, a gallium arsenide nanostructure, a doped silicon nanostructure, an aluminum nanostructure, a magnesium nanostructure, a silver and gold composite nanostructure, and combinations thereof.

26. The labeled nucleotide as defined in claim 24, wherein the plasmonic nanostructure is attached to the base of the nucleotide through a double stranded deoxyribonucleic acid strand.

27. The labeled nucleotide as defined in claim 24, wherein:
a first linking molecule attaches the dye label to the base of the nucleotide;
a second linking molecule attaches the plasmonic nanostructure to the base of the nucleotide; and
the first linking molecule has a first length that is within from about 3 nm to about 12 nm of a second length of the second linking molecule.

28. The labeled nucleotide as defined in claim 24, wherein:
a first linking molecule attaches the dye label to the base of the nucleotide;
a second linking molecule attaches the plasmonic nanostructure to the base of the nucleotide; and
the first linking molecule has a first length, the second linking molecule has a second length, and together the first and second lengths range from about 3 nm to about 12 nm.

29. A method, comprising:
introducing an incorporation mix to a flow cell including clusters of template strands, the incorporation mix including:
a liquid carrier;
a plurality of polymerases; and
a plurality of labeled nucleotides, each labeled nucleotide including:
a nucleotide;
a 3′ OH blocking group attached to a sugar of the nucleotide;
a dye label attached to a base of the nucleotide; and
a plasmonic nanostructure attached to the base of the nucleotide;
whereby at least one the polymerases incorporates an individual one of the labeled nucleotides into a nascent strand along one the template strands; and
optically imaging the incorporation.
